(19) United States
(12) Patent Application Publication
(10) Pub. No.: US 2024/0041011 A1
(43) Pub. Date: Feb. 8, 2024

(54) INFECTIOUS DISEASE MOUSE MODELS

(71) Applicant: The Jackson Laboratory, Bar Harbor, ME (US)

(72) Inventor: James Keck, Granite Bay (CA)

(73) Assignee: The Jackson Laboratory, Bar Harbor, ME (US)

(21) Appl. No.: 18/265,521

(22) PCT Filed: Dec. 6, 2021
(86) PCT No.: PCT/US2021/062007

§ 371 Date: Jun. 6, 2023

Related U.S. Application Data
(60) Provisional application No. 63/122,416, filed on Dec. 7, 2020.

(51) Int. Cl. A01K 67/027; C12N 5/071; C12N 5/09

(52) U.S. Cl. CPC A01K 67/0271; C12N 5/0688; C12N 5/0693; A01K 2207/12; A01K 2207/15; A01K 2227/105; A01K 2267/0337

(57) ABSTRACT

Provided herein are immnunodeficient mouse models engrafted with cells comprising a pathogen entry moiety, for example, for assessing pathogenic infection. The pathogen may be a virus, such as a respiratory virus.

### RELATED APPLICATION

This application claims the benefit under 35 U.S.C. 119(e) of the filing date of U.S. Provisional Application Ser. No. 63/122,416, filed Dec. 7, 2020, the entire content of which is incorporated herein by reference.

### BACKGROUND

The world is currently facing one of the worst medical emergencies since WWII: the coronavirus disease 2019 (COVID-19) pandemic caused by infection with a novel coronavirus, referred to as SARS-CoV-2. The lack of timely action is in part due to the absence of an appropriate animal model to use to understand the viral infection and help test therapeutics. The scientific community has recently made significant advancements using mice genetically engineered to express key human proteins. However, knowledge of the essential biological mechanisms is important for this approach to be successful. Given the molecular complexities surrounding host-pathogen interactions, genetically engineering and characterizing a robust animal model (with all key targets appropriately humanized) for a novel disease like COVID-19 will take up to a year or more.

### SUMMARY

The present disclosure provides, in some aspects, humanized immunodeficient mouse models engrafted with human cancer cells, for example, from a patient derived xenograft (PDX), that contain human molecular and cellular factors required for pathogenic infection. Many of the human molecular and cellular factors required for pathogenic infection are expressed at varying levels in different PDX tumor models. By engrafting humanized immunodeficient mice, for example, with PDX tumor tissue and exposing the mice to one of a myriad of pathogens, the infectious disease community now has a robust model for studying the complexities of pathogenesis and for testing candidate substances for combating pathogenesis. These humanized immunodeficient mouse models advance the field by providing model systems that more accurately replicate the human immune system, including its vulnerabilities and defense mechanisms associated with pathogenic infection and disease progression.

Some aspects of the present disclosure provide an immunodeficient mouse engrafted with cells comprising a host cell moiety associated with pathogenesis (“host cell moiety”), wherein the mouse is infected with a pathogen comprising a surface moiety that binds to the host cell moiety.

In some embodiments, the cells comprise mammalian cells. The mammalian cells may comprise, for example, human cells, non-human primate cells, or canine cells. In some embodiments, the mammalian cells are human cells.

In some embodiments, the cells are cancer cells.

In some embodiments, the cells are immortalized cells.

In other embodiments, the cells are primary cells.

In some embodiments, the cells are from a PDX.

In some embodiments, the cells are genetically engineered to alter sensitivity of the cells to a pathogen.

In some embodiments, the cells are genetically engineered to express a detectable biomolecule, optionally a fluorescent or bioluminescent protein.

In some embodiments, lung tissue, liver tissue, gastrointestinal tissue, brain tissue, skin tissue, and/or bone marrow of the mouse is engrafted with the cells.

In some embodiments, the mouse has a non-obese diabetic (NOD) genotype.

In some embodiments, the mouse comprises a null mutation in a murine Prkdc gene.

In some embodiments, the mouse comprises a null mutation in a murine Il2rg gene.

In some embodiments, the mouse has a NOD scid gamma genotype.

In some embodiments, the cells are from a bladder tumor, brain tumor, breast tumor, colon and rectal tumor, endometrial tumor, kidney tumor, leukemia, liver tumor, lung tumor, melanoma, non-Hodgkin lymphoma, ovarian tumor, pancreatic tumor, prostate tumor, sarcoma, skin tumor, testicular tumor or thyroid tumor.

In some embodiments, the mouse is engrafted with human peripheral blood mononuclear cells (PMBCs) or human hematopoietic stem cells (HSCs).

In some embodiments, the PMBCs or HSCs are HLA-matched to the human PDX. In other embodiments, the PMBCs or HSCs are non-HLA-matched to the human PDX.

In some embodiments, the host cell moiety is selected from membrane proteins, lipids, and carbohydrate moieties, optionally present either on glycoproteins or glycolipids.

In some embodiments, the surface protein is selected from proteins, glycans, and lipids.

In some embodiments, the pathogen is selected from bacteria, viruses, prions, and fungi.

In some embodiments, the pathogen is a virus.

In some embodiments, the virus is a respiratory virus.

In some embodiments, the respiratory virus is selected from influenza viruses, respiratory syncytial viruses, parainfluenza viruses, adenoviruses, and coronaviruses.

In some embodiments, the respiratory virus is a coronavirus, optionally an alphacoronavirus or a beta coronavirus.

In some embodiments, the coronavirus is selected from 229E, NL631 OC43, HKU1, MERS-CoV, SARS-CoV, and SARS-CoV-2. The coronavirus may be, for example, SARS-CoV-2.

In some embodiments, the surface protein is a surface glycoprotein, optionally a spike (S) protein.

In some embodiments, the pathogen entry moiety is a protein selected from ACE2, CD147, and TMPRSS2.

In other embodiments, the virus is a Flaviviridae virus, optionally selected from yellow fever virus, West Nile virus, Dengue virus, Zika virus, and Powassan virus.

In yet other embodiments, the virus is a Togaviradae virus, optionally Chikungunya virus.

In still other embodiments, the virus is a Bunyaviriade virus, optionally a LaCrosse virus.

Other aspects of the present disclosure provide a method comprising administering to an immunodeficient mouse cells of a PDX, wherein the cells comprise a pathogen entry moiety, and administering to the immunodeficient mouse a pathogen comprising a surface moiety that binds to the pathogen entry moiety.

In some embodiments, the cells are administered sample of the single cell suspension is delivered by In some embodiments, the administering is by tail vein injection, cardiac injection, caudal artery injection, cranial injection, hepatic artery injection, femoral injection, peritoneal injection, or tibial injection.

In some embodiments, the method further comprises delivering to the mouse a therapeutic agent or a prophylactic agent.

In some embodiments, the method further comprises assessing toxicity of the agent.

In some embodiments, the method further comprises assessing efficacy of the agent for treating or preventing infection by the virus and/or development of a disease caused by the pathogen.

In some embodiments, the method further comprises assessing one or more of the following: viral load in the mouse; viral titer in the mouse; respiratory function and/or cardiac function of the mouse; the human immune cell-mediated response to the virus; and a biomarker of viral disease progression.

Yet other aspects of the present disclosure provide method comprising: infecting multiple human cell samples with a pathogen, wherein each of the samples comprises human cells from a different source; measuring viral load for each of the samples; and identifying a sample having a viral load greater than a reference value.

In some embodiments, the different sources are different PDXs.

In some embodiments, the different sources are different human subjects.

In some embodiments, the different sources are different cell lines.

In some embodiments, the method further comprises delivering to an immunodeficient mouse cells of the sample having a viral load greater than a reference value.

In some embodiments, the method further comprises assessing genetic differences among the human cells of the samples.

In some embodiments, the method further comprises genetically mapping genes of the human cells necessary for pathogenic infection.

In some embodiments, the method further comprises identifying at least one pathogen entry moiety used by the pathogen for entry into the human cells.

In some embodiments, the method further comprises administering to the immunodeficient mouse the pathogen.

In some embodiments, the method further comprises administering to the immunodeficient mouse a therapeutic agent or a prophylactic agent.

In some embodiments, wherein the method is performed in vitro.

### BRIEF DESCRIPTION OF THE DRAWINGS

FIG. 1. In vivo imaging system. Shown is a Xenogen image of NSG™ mice 14 days after intravenous (IV) injection of human breast adenocarcinoma cells expressing luciferase (MDA-MB-231-Luc cells). The majority of luciferase signal is found in the lungs.

FIGS. 2A-2B show the results of a qPCR assay to detect human ACE2 mRNA in naïve and humanized mice (FIG. 2A) and the results of a flow cytometry analysis to detect human cell surface proteins in naïve and humanized mice (FIG. 2B). Signals were normalized to mouse GAPDH mRNA.

FIGS. 3A-3B show qPCR results showing SARS-CoV-2 mRNA levels (FIG. 3A) and human ACE2 (hACE2) levels in PDX-humanized mice after intranasal (IN) or intravenous (IV) infection with SARS-CoV-2 compared to uninfected (Un) mice. Signals were normalized to mouse GAPDH mRNA.

FIG. 4 is a qPCR analysis showing the SARS-CoV-2 mRNA expression levels in different organs of naïve mice (open shapes) and PDX-humanized mice (closed shapes). All signals are normalized to mouse GAPDH.

FIG. 5 is a qPCR analysis showing the SARS-CoV-2 mRNA expression levels in different organs of naïve mice (gray circles) and HuH7.5-Luc humanized mice (black circles).

FIGS. 6A-6B show the results of a qPCR assay to detect human ACE2 mRNA expression levels in PDX tumors from uninfected mice humanized with different cell lines (FIG. 6A) and compared to k18 transgenic mouse lung samples, which are known to be susceptible to SARS-CoV-2 (FIG. 6B). **, p<0.01.

FIG. 7 shows the SARS-CoV-2 infectious virus, measured as focus forming units per millieter (FFU/mL) in NSG mice and NSG PDX TM219 mice two days after infection.

FIGS. 8A-8B show a comparison of SARS-CoV-2 infections in NSG mice humanized with different PDXs. The levels of infectious virus (FIG. 8A) and genome copies of the virus (FIG. 8B) were determined. *, p<0.05; **, p<0.01.

FIGS. 9A-9B show the viral load (FIG. 9A) and viral titer (FIG. 9B) of SARS-CoV-2 in the lungs of NSG and NSG-PDX mice two (2 dpi) and four (4 dpi) days post-infection.

FIGS. 10A-10B show the results of in vitro studies of PDX: the SARS-CoV-2 viral RNA levels (FIG. 10A) and the SARS-CoV-2 focus forming assay (FIG. 10B) in three PDX tumor cell lines.

FIG. 11 shows the results of an in vitro study of PDX, examining influenza A viral RNA expression in three PDX tumor cell lines.

FIGS. 12A-12E show the results of in vitro experiments where primary cell lines were infected with different viruses and levels of expression relative to GAPDH were determined. The viruses tested were: West Nile virus (FIG. 12A), Langat virus (FIG. 12B), Powassan LB and Spooner virus (FIG. 12C), Chikungunya virus (FIG. 12D), and LaCrosse virus (FIG. 12E).

FIGS. 13A-13B show the results of in vitro experiments described in FIGS. 12A-12E) at two timepoints: 24 hours post-infection (FIG. 13A) and 48 hours post-infection (FIG. 13B). The viruses tested, from left to right, were: West Nile virus (WNV), Powassan LB virus (LB) and Powassan Spooner virus (Spo), Langat virus (LGTV), LaCrosse virus (LACV), and Chikungunya virus (CHIKV),

### DETAILED DESCRIPTION

The present disclosure provides, in some embodiments, humanized immunodeficient mouse models, methods of producing the models, and methods of using the models, for example, to assess pathogen infection and disease. The models provided herein are engrafted with cells, such as mammalian (e.g., human) cells, for example, human cells cancer cells, for example, from a patient-derived xenograft (PDX) or a human cancer cell line. Cancerous cells and tissues are associated with unique genetic profiles that are used herein to produce mouse models that can be utilized, for example, to explore the genetic components that may underly and/contribute to host susceptibility to pathogen infection and related disease progression. By replicating the human immune system responses to pathogenic infection, these models may also be used in some embodiments to evaluate toxicity (e.g., cytokine release syndrome) and other side effects of certain therapeutics aimed at preventing or treating pathogenic disease. In some embodiments, the mouse models described herein may be used to determine the genes necessary for an unknown pathogen's transmissibility or infectability. For instance, if a new variant of a known virus or a new virus is found, the methods can be used to determine which receptor (or receptors) is used for infection of humans. This is demonstrated in Example 3 with five different viruses from three different families and a panel of genetically diverse PDXs or PDX-derived cell lines. By assessing at the resulting viral loads after infection in vitro, the genetic similarities between the PDXs that enable infection can be investigated to determine the critical genes and proteins necessary for infection. The most effective PDX or PDX-derived cell lines can then be used to build a model for studying the new pathogen and helping to find a new therapeutic or vaccine, as described herein.

An animal model may be, but is not limited to, a non-human mammal, a rodent (e.g., a mouse, a rat, or a hamster), or a livestock animal (e.g., a pig, a cow, a chicken, or a goat) model. In some embodiments, the animal is a rodent. In some embodiments, the animal is a mouse.

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

A mouse model of pathogenic disease may be modified to enable the assessment of a pathogen or pathogenic disease. Any system (e.g., immune, respiratory, nervous, or circulatory), organ (e.g., blood, heart, blood vessels, spleen, thymus, lymph nodes, or lungs), tissue (e.g., epithelial, connective, muscle, and nervous), or cell type (e.g., lymphocytes or macrophages) may be modified, either independently or in combination, to enable studying a pathogen or pathogenic disease in the models provided herein.

### Immunodeficient Mouse Models

Provided herein, in some embodiments, are immunodeficient mouse models. As is known in the art, immunodeficient mice have impaired or disrupted immune systems, such as specific deficiencies in MHC class I, II or both, B cell or T cell defects, or defects in both, natural killer cell defects, myeloid defects (e.g., defects in granulocytes and/or monocytes), as well as immunodeficiency due to knockdown of genes for cytokines, cytokine receptors, TLR receptors and a variety of transducers and transcription factors of signaling pathways. Immunodeficiency mouse models include the single-gene mutation models such as nude-mice (nu) strains and the severe combined immunodeficiency (scid) strains, non-obese diabetic (NOD) strain, RAG (recombination activating gene) strains with targeted gene deletion and a variety of hybrids originated by crossing doubly and triple mutation mice strains with additional defects in innate and adaptive immunity.

Non-limiting examples of spontaneous and transgenic immunodeficient mouse models include the following mouse strains:
Nude (nu) [Flanagan S P. Genet Res 1966; 8: 295-309; and Nehls M et al. Nature 1994; 372: 103-7];
Scid (scid) [Bosma G C et al. Nature 1983; 301:527-30; Mosier D E et al. Nature 1988; 335: 256-9; and Greiner D L et al. Stem Cells 1998; 16: 166-77];
NOD [Kikutani H et al. Adv Immunol 1992; 51: 285-322; and Anderson M S et al. Ann Rev Immunol 2005; 23: 447-85];
RAG1 and RAG2 (rag) [Mombaerts P et al. Cell 1992; 68: 869-77; Shinkai U et al. Cell 1992; 68: 855-67];
NOD-scid [Greiner D L et al. 1998; Shultz L D et al. J Immunol 1995; 154: 180-91; Melkus M W et al. Nature Med 2006; 12: 1316-22; and Denton P W et al. PLoS Med 2008; 4(12): e357];
IL2rgnull [DiSanto J P et al. Proc Natl Acad Sci USA 1995; 92: 377-81];
B2mnull [Christianson S W et al. J Immunol 1997; 158: 3578-86];
NOD-scid IL2rγnull [Shultz L D et al. Nat Rev Immunol 2007; 7: 118-30; Ito M et al. Blood 2002; 100: 3175-82; Ishikawa I et al. Blood 2005; 106: 1565-73; and Macchiarini F et al. J Exp Med 2005; 202: 1307-11];
NOD-scid B2mnull [Shultz et al. 2007; Shultz L D et al. Transplantation 2003; 76: 1036-42; Islas-Ohlmayer M A et al. J Virol 2004; 78:13891-900; and Macchiarini et al. 2005];
HLA transgenic mice [Grusby M J et al. Proc Natl Acad Sci USA 1993; 90(9): 3913-7; and Roy C J et al. Infect Immun 2005; 73(4): 2452-60]. See, e.g., Belizario J E The Open Immunology Journal, 2009; 2:79-85;
NOG mice (NOD.cg-Prkdc^{scid}Ilm2rg^{rm1Stg}) [Shultz L D et al. Nat Rev Immunol 2007; 7: 118-30]; and
BRG mice (BALB/c; 129S4-Rag2^{tm1.1Flv}) [Shultz L D et al. Nat Rev Immunol 2007; 7: 118-30].

Provided herein, in some embodiments, are immunodeficient mouse models having the non-obese diabetic (NOD) mouse genotype. The NOD mouse (e.g., Jackson Labs Stock #001976, NOD-Shi^{LtJ}) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique NMC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement, C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background may have a genotype selected from NOD-Cg.-Prkdc^{scid}IL2rg^{tm1wJl}/SzJ (NSG™), a NOD.Cg-Rag1^{tm1Mom} Il2rg^{t11Wj6}/SzJ (NRG), and NOD Cg-Prkdc^{scid}Il2rg^{tm1Sug}/ShiJic (NOG). Other immunodeficient mouse strains are contemplated herein.

In some embodiments, an immunodeficient mouse model has an NSG™ genotype. The NSG™ mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG™ mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2^{tm1Wj1}targeted mutation. The Prkdc^{scid} mutation is a loss-of-function (null) mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1Wj1} an mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}) The Rag1^{null} mutation renders the mice B and T cell deficient and the IL2rg^{null} mutation prevents cytokine signaling through multiple receptors, leading to a deficiency in functional NK cells. The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-7 chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the l12rg gene further exacerbates the SCID-like phenotype while additionally resulting in a decrease of NK cell production.

Provided herein, in some embodiments, are immunodeficient mouse models that are deficient in MHC Class I, MHC Class 11, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., α chain and β chain) or does not have the same level of MHC Class I and/or MHC Class II protein activity as a non-immunodeficient (e.g., MHC Class I/II wild-type) mouse. In some embodiments, the expression or activity of MHC Class I and/or MHC Class II proteins is reduced (e.g., by at least 30%, at least 40%, at least 50%, at least 60%, at least 70%, at least 80%, at least 90% or more), relative to a non-immunodeficient mouse.

Immunodeficient mice that are deficient in MHC Class I, MHC Class II, and MHC Class I and MHC Class II are described in International Publication No. WO 2018/209344, the contents of which are incorporated by reference herein.

The terms “MHC Class I” and “MHC I” are used interchangeably herein and refer to a complex formed by MHC I α protein and β2-microglobulin. MHC Class I α proteins includes an extracellular domain with the subdomains α1, α2, and α3, a transmembrane domain, and a cytoplasmic tail. The terms “H2-K”, “H2-D”, and “H2-L” refer to MHC Class I α protein subclasses, all of which are encoded on mouse chromosome 17. β2-microglobulin associates noncovalently with the α3 subdomain of MHC I α protein. The gene encoding mouse β2-microglobulin is encoded on mouse chromosome 2.

The terms “MHC Class II” and “MHC II” are used interchangeably to refer to a complex formed by two non-covalently associated proteins: an MHC II α protein and an MHC II R protein. The terms “H-2A” and “H-2E” (often abbreviated as I-A and I-E, respectively) refer to subclasses of MHC II. The MHC II α protein and MHC II β proteins span the plasma membrane and each contains an extracellular domain, a transmembrane domain, and a cytoplasmic domain. The extracellular portion of the MHC II α protein includes MHC II α1 and MHC II α2 domains, and the extracellular portion of the MHC II β protein includes MHC II β1 and MHC II β2 domains.

In some embodiments, an immunodeficient mouse deficient in MHC class I and MHC class II is a NOD.Cg-Prkdc^{scid}H2K1^{tm1Bpe} H2-Ab1^{em1Mvw} H2-D1^{tm1Bpe} Il2rgr^{tm1Wj1}/SzJ (abbreviated as NSG-(K^{b} D^{b})^{null} (IA^{null})). The NSG-(K^{b} D^{b})^{null} (IA^{null}) mouse lacks functional MHC I due to a homozygous null mutation of H2-K and H2-D MHC I α protein subclasses (abbreviated (K^{b} D^{b})^{null}). The NSG-(K^{b} D^{b})^{null} (IA^{null}) mouse lacks functional MHC II due to a homozygous null mutation of H-2A subclass of MHC II (abbreviated as A^{null}).

In some embodiments, an immunodeficient mouse deficient in MHC class I and MHC class II is a NOD.Cg-Prkdc^{scid} H2-K1^{tm1Bpe} H2-Ab1^{em1Mvw} H2-D1^{tm1Bpe} Il2rg^{tm1Wj1} Tg(Ins2-HBEGF)6832Ugfm/Sz (abbreviated as NSG-B2MP^{null} (IA IE)^{null}) mouse. The NSG-B2M^{null} (IA IE)^{null} mouse lacks functional MHC I due to a homozygous null mutation of β2 microglobulin (abbreviated B2MP^{null}). The NSG-B2M^{null} (IA IE)^{null} mouse lacks functional MHC II due to a homozygous null mutation of H-2A and H-2E subclasses of MHC II (abbreviated as (IA IE)^{null}).

In some embodiments, an immunodeficient mouse deficient in MHC class I and MHIIC class II is a NOD.Cg-Prkdc^{scid} H2-K1^{tm1Bpe} H2-Ab1^{em1Mvw} H2-D1^{tm1Bpe} Il2rg^{tm1Wj1} Tg(Ins2-HIBEGF)6832Ugfm/Sz transgenic mouse, abbreviated as NSG-RIP-DTR (K^{b} D^{b})^{null} (IA^{null}), which expresses the diphtheria toxin receptor under the control of the rat insulin promoter on an NSG™ background. Injection of diphtheria toxin (DT) into mice expressing the diphtheria toxin receptor under the control of the rat insulin promoter leads to mouse pancreatic beta cell death and hyperglycemia. The NSG-RIP-DTR (K^{b} D^{b})^{null} (IA^{null}) strain permits the complete and specific ablation of mouse pancreatic beta cells, avoiding the broadly toxic effects of diabetogenic drugs such as streptozotocin.

### Humanized Mouse Models

Provided herein, in some embodiments, are humanized immunodeficient mouse models and methods of producing the models. Immunodeficient mice engrafted with functional human cells and/or tissues are referred to as “humanized mice.” As used herein, the terms “humanized mouse”, “humanized immune deficient mouse”, “humanized immunodeficient mouse”, and the plural versions thereof are used interchangeably to refer to an immunodeficient mouse humanized by engraftment with functional human cells and/or tissues. For example, mouse models may be engrafted with human hematopoietic stem cells (HSCs) and/or human peripheral blood mononuclear cells (PMBCs). In some embodiments, mouse models are engrafted with human tissues such as islets, liver, skin, and/or solid or hematologic cancers. In other embodiments, mouse models may be genetically modified such that endogenous mouse genes are converted to human homologs (see, e.g., Pearson, et al., Curr Protoc Immunol., 2008, Chapter: Unit—15.21).

Humanized mice are generated by starting with an immunodeficient mouse and, if necessary, depleting and/or suppressing any remaining murine immune cells (e.g., chemically or with radiation). That is, successful survival of the human immune system in the immunodeficient mice may require suppression of the mouse's immune system to prevent GVHD (graft-versus-host disease) rejections. After the immunodeficient mouse's immune system has been sufficiently suppressed, the mouse is engrafted with human cells (e.g., HSCs and/or PBMCs). As used herein, “engraft” refers to the process of the human cells migrating to, and incorporating into, an existing tissue of interest in vivo. With respect to the humanized immunodeficient mouse, the engrafted human cells provide functional mature human cells (e.g., immune cells). The model has a specific time window of about 4-5 weeks after engraftment before GVHD sets in. To increase the longevity of the model, double-knockout mice lacking functional MHC I and MHC II, as described above, may be used.

The engrafted human cells (e.g., HSCs or PMBCs) for humanization, in some embodiments, are human leukocyte-antigen (HLA)-matched to the human cancer cells of the mouse models. HLA-matched refers to cells that express the same major histocompatibility complex (MHC) genes. Engrafting mice with HLA-matched human xenografts and human immune cells, for example, reduces or prevents immunogenicity of the human immune cells. In some embodiments, a humanized mouse provided in the present disclosure is engrafted with human PMBCs or human HSCs that are HLA-matched to a PDX or human cancer cell line.

The engrafted human cells (e.g., HSCs or PMBCs) for humanization, in some embodiments, are not HLA-matched to the human cancer cells of the mouse models. That is, in some embodiments, a humanized mouse provided in the present disclosure is engrafted with human PMBCs or human HSCs that are not HLA-matched to a PDX or human cancer cell line.

Myeloablation

As described above, in some embodiments, immunodeficient mice are treated to deplete and/or suppress any remaining murine immune cells (e.g., chemically and/or with radiation). In some embodiments, immunodeficient mice are treated only chemically or only with radiation. In other embodiments, immunodeficient mice are treated both chemically and with radiation.

In some embodiments, immunodeficient mice are administered a myeloablative agent, that is, a chemical agent that suppresses or depletes murine immune cells. Examples of myeloablative agents include busulfan, dimethyl mileran, melphalan, and thiotepa.

In some embodiments, immunodeficient mice are irradiated prior to engraftment with human cells, such as human HSCs and/or PMBCs. It is thought that irradiation of an immunodeficient mouse destroys mouse immune cells in peripheral blood, spleen, and bone marrow, which facilitates engraftment of human cells, such as human HSCs and/or PMBCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG™ mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

A myeloablative irradiation dose is usually 700 to 1300 cGy, though in some embodiments, lower doses such as 1-100 cGy (e.g., about 2, 5, or 10 cGy), or 300-700 cGy may be used.

As an example, the mouse may be irradiated with 100 cGy X-ray (or 75 cGy-125 cGy X-ray). In some embodiments, the dose is about 1, 2, 3, 4, 5, 10, 20, 100, 200, 300, 400, 500, 600, 700, 800, 900, 1000, 1100, 1200, or 1300 cGy, or between any of the two recited doses herein, such as 100-300 cGy, 200-500 cGy, 600-1000 cGy, or 700-1300 cGy. In some embodiments, the immunodeficient mouse is irradiated about 15 minutes, 30 minutes, 45 minutes, 1 hour, or more before engraftment with human HSCs and/or PMBCs. In some embodiments, the immunodeficient mouse is engrafted with human HSCs and/or PMBCs 1, 2, 3, 4, 5, 6, 7, 8, 9, 10, 11, 12, 13, 14, 15, 16, 17, or 18 days after irradiation.

Engraftment

As described above, in some embodiments, the irradiated immunodeficient mice are engrafted with HSCs and/or PBMCs, humanizing the mice. Engraftment refers to the process of the human cells migrating to, and incorporating into, an existing tissue of interest in vivo. The PBMCs may be engrafted after irradiation and before engraftment of human cancer cells, after irradiation and concurrently with engraftment of human cancer cells, or after irradiation and after engraftment of human cancer cells.

PBMCs

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of mononuclear cells, lymphocytes and monocytes. The lymphocyte population of PBMCs typically includes T cells, B cells and NK cells.

PBMCs may be isolated from whole blood samples, for example (e.g., Ficoll gradient). In some embodiments, PBMCs from a subject (e.g., a human subject) with a current or previous diagnosis of a pathogen or pathogenic disease may be used.

HSCs

Hematopoietic stem cells (HSCs) are the stem cells that give rise to other blood cells during a process referred to as hematopoiesis. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells.

Methods of engrafting immunodeficient mice with HSCs and/or PBMCs to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32(2): 194-2020; Yaguchi et al., Cell & Mol Immunol, 2018, 15:953-962). In some embodiments, the mouse is engrafted with 1.0×10⁶-3.0×10⁷ HSCs and/or PBMCs.

For example, the mouse may be engrafted with 1.0×10⁶, 1.1×10⁶, 1.2×10⁶, 1.3×10⁶, 1.4 10×10⁶, 1.5×10⁶, 1.6×10⁶, 1.7×10⁶, 1.8×10⁶, 1.9×10⁶, 2.0×10⁶, 2.5×10⁶, 3.0×10⁶ or more HSCs and/or PBMCs. In some embodiments, the mouse is engrafted with 1.0-1.1×10⁶, 1.0-1.2×10⁶, 1.0-1.3×10⁶, 1.0-1.4×10⁶, 1.0-1.5×10⁶, 1.0-1.6×10⁶, 1.0-1.7×10⁶, 1.0-1.8×10⁶, 1.0-1.9×10⁶, 1.0-2.0×10⁶, 1.0-2.25×10⁶, 1.0-2.5×10⁶, 1.0-2.75×10⁶, 1.0-3.0×10⁶, 1.1-1.2×10⁶, 1.1-1.3×10⁶, 1.1-1.4×10⁶, 1.1-1.5×10⁶, 1.1-1.6×10⁶, 1.1-1.7×10⁶, 1.1-1.8×10⁶, 1.1-1.9×10⁶, 1.1-2.0×10⁶, 1.1-2.25×10⁶, 1.1-2.5×10⁶, 1.1-2.75×10⁶, 1.1-3.0×10⁶, 1.2-1.3×10⁶, 1.2-1.4×10⁶, 1.2-1.5×10⁶, 1.2-1.6×10⁶, 1.2-1.7×10⁶, 1.2-1.8×10⁶, 1.2-1.9×10⁶, 1.2-2.0×10⁶, 1.2-2.25×10⁶, 1.2-2.5×10⁶, 1.2-2.75×10⁶, 1.2-3.0×10⁶, 1.3-1.4×10⁶, 1.3-1.5×10⁶, 1.3-1.6×10⁶, 1.3-1.7×10⁶, 1.3-1.8×10⁶, 1.3-1.9×10⁶, 1.3-2.0×10⁶, 1.3-2.25×10⁶, 1.3-2.5×10⁶, 1.3-2.75×10⁶, 1.3-3.0×10⁶, 1.4-1.5×10⁶, 1.4-1.6×10⁶, 1.4-1.7×10⁶, 1.4-1.8×10⁶, 1.4-1.9×10⁶, 1.4-2.0×10⁶, 1.4-2.25×10⁶, 1.4-2.5×10⁶, 1.4-2.75×10⁶, 1.4-3.0×10⁶, 1.5-1.6×10⁶, 1.5-1.7×10⁶, 1.5-1.8×10⁶, 1.5-1.9×10⁶, 1.5-2.0×10⁶, 1.5-2.25×10⁶, 1.5-2.5×10⁶, 1.5-2.75×10⁶, 1.5-3.0×10⁶, 1.6-1.7×10⁶, 1.6-1.8×10⁶, 1.6-1.9×10⁶, 1.6-2.0×10⁶, 1.6-2.25×10⁶, 1.6-2.5×10⁶, 1.6-2.75×10⁶, 1.6-3.0×10⁶, 1.7-1.8×10⁶, 1.7-1.9×10⁶, 1.7-2.0×10⁶, 1.7-2.25×10⁶, 1.7-2.5×10⁶, 1.7-2.75×10⁶, 1.7-3.0×10⁶, 1.8-1.9×10⁶, 1.8-2.0×10⁶, 1.8-2.25×10⁶, 1.8-2.5×10⁶, 1.8-2.75×10⁶, 1.8-3.0×10⁶, 1.9-2.0×10⁶, 1.9-2.25×10⁶, 1.9-2.5 25×10⁶, 1.9-2.75×10⁶, 1.9-3.0×10⁶, 2.0-2.25×10⁶, 2.0-2.5×10⁶, 2.0-2.75×10⁶, 2.0-3.0×10⁶, 2.25-2.5×10⁶, 2.25-2.75×10⁶, 2.25-3.0×10⁶, 2.5-2.75×10⁶, 2.5-3.0×10⁶, or 2.75-3.0×10⁶ HSCs and/or PBMCs.

In some embodiments, the mouse may be engrafted with 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.5×10⁷, 3.0×10⁷ or more HSCs and/or PBMCs. In some embodiments, the mouse is engrafted with 1.0-1.1×10⁷, 1.0-1.2×10⁷, 1.0-1.3×10⁷, 1.0-1.4×10⁷, 1.0-1.5×10⁷, 1.0-1.6×10⁷, 1.0-1.7×10⁷, 1.0-1.8×10⁷, 1.0-1.9×10⁷, 1.0-2.0×10⁷, 1.0-2.25×10⁷, 1.0-2.5×10⁷, 1.0-2.75×10⁷, 1.0-3.0×10⁷, 1.1-1.2×10⁷, 1.1-1.3×10⁷, 1.1-1.4×10⁷, 1.1-1.5×10⁷, 1.1-1.6×10⁷, 1.1-1.7×10⁷, 1.1-1.8×10⁷, 1.1-1.9×10⁷, 1.1-2.0×10⁷, 1.1-2.25×10⁷, 1.1-2.5×10⁷, 1.1-2.75×10⁷, 1.1-3.0×10⁷, 1.2-1.3×10⁷, 1.2-1.4×10⁷, 1.2-1.5×10⁷, 1.2-1.6×10⁷, 1.2-1.7×10⁷, 1.2-1.8×10⁷, 1.2-1.9×10⁷, 1.2-2.0×10⁷, 1.2-2.25×10⁷, 1.2-2.5×10⁷, 1.2-2.75×10⁷, 1.2-3.0×10⁷, 1.3-1.4×10⁷, 1.3-1.5×10⁷, 1.3-1.6×10⁷, 1.3-1.7×10⁷, 1.3-1.8×10⁷, 1.3-1.9×10⁷, 1.3-2.0×10⁷, 1.3-2.25×10⁷, 1.3-2.5×10⁷, 1.3-2.75×10⁷, 1.3-3.0×10⁷, 1.4-1.5×10⁷, 1.4-1.6×10⁷, 1.4-1.7×10⁷, 1.4-1.8×10⁷, 1.4-1.9×10⁷, 1.4-2.0×10⁷, 1.4-2.25×10⁷, 1.4-2.5×10⁷, 1.4-2.75×10⁷, 1.4-3.0×10⁷, 1.5-1.6×10⁷, 1.5-1.7×10⁷, 1.5-1.8×10⁷, 1.5-1.9×10⁷, 1.5-2.0×10⁷, 1.5-2.25×10⁷, 1.5-2.5×10⁷, 1.5-2.75×10⁷, 1.5-3.0×10⁷, 1.6-1.7×10⁷, 1.6-1.8×10⁷, 1.6-1.9×10⁷, 1.6-2.0×10⁷, 1.6-2.25×10⁷, 1.6-2.5×10⁷, 1.6-2.75×10⁷, 1.6-3.0×10⁷, 1.7-1.8×10⁷, 1.7-1.9×10⁷, 1.7-2.0×10⁷, 1.7-2.25×10⁷, 1.7-2.5×10⁷, 1.7-2.75×10⁷, 1.7-3.0×10⁷, 1.8-1.9×10⁷, 1.8-2.0×10⁷, 1.8-2.25×10⁷, 1.8-2.5×10⁷, 1.8-2.75×10⁷, 1.8-3.0×10⁷, 1.9-2.0×10⁷, 1.9-2.25×10⁷, 1.9-2.5×10⁷, 1.9-2.75×10⁷, 1.9-3.0×10⁷, 2.0-2.25×10⁷, 2.0-2.5×10⁷, 2.0-2.75×10⁷, 2.0-3.0×10⁷, 2.25-2.5×10⁷, 2.25-2.75×10⁷, 2.25-3.0×10⁷, 2.5-2.75×10⁷, 2.5-3.0×10⁷, or 2.75-3.0×10⁷ HSCs and/or PBMCs. In some embodiments, the mouse is engrafted with 2×10⁷ HSCs and/or PBMCs. According to some embodiments, the mouse is engrafted with 4.5-5.5×10⁷ (4.5-5.0×10⁷, 5.0-5.5×10⁷) HSCs and/or PBMCs.

### Mammalian Cell Lines and Patient-Derived Xenografts

The mouse models of pathogenic disease provided herein, in some embodiments, are engrafted with cells (viable cells), for example, mammalian cells, such as those from a cell line or a patient (e.g., a human or canine patient-derived xenograft). As used herein, “mammal” includes, but is not limited to, mice, rats, rabbits, guinea pigs, dogs, cats, sheep, goats, cows, horses, primates such as monkeys, chimpanzees and monkeys, and, in particular, humans. In some embodiments, a mouse model is engrafted with human cells. In other embodiments, a mouse model is engrafted with canine cells.

The cells may be diseased cells, in some embodiments, for example, cancer cells. Other diseased cells are contemplated herein (e.g., those cells obtained from a patient having a cardiovascular disease, metabolic disease, autoimmune disease, etc.).

In some embodiments, the mouse models are engrafted with human cancer cells. The human cancer cells may be from a single source or from multiple sources (e.g., at least 2, at least 3, at least 4, at least 5, at least 6, at least 7, at least 8, at least 9, or at least 10 sources). The human cancer cells may be tumor cells (e.g., cells from a malignant tumor), patient-derived xenografts (PDXs) (e.g., tumor tissue from a human that is implanted in a mouse model), or human cancer cell lines (e.g., human cancer cell cultures developed from a single cell). A tumor is a mass of tissue formed by the abnormal growth of cells (e.g., cancer cells). Non-limiting examples of common human cancers include bladder cancer, brain cancer, breast cancer, colon and rectal cancer, endometrial cancer, kidney cancer, leukemia, liver cancer, lung cancer, melanoma, non-Hodgkin lymphoma, ovarian cancer, pancreatic cancer, prostate cancer, sarcoma, skin cancer, testicular cancer, and thyroid cancer. Other cancer cell types are contemplated herein (see, e.g., cancer.gov/types).

In some embodiments, human cancer cells may be circulating tumor cells, for example, from a primary tumor or a secondary tumor. A primary tumor is a tumor growing at the anatomical site where the tumor originated (e.g., lung cancer tumor or breast cancer tumor). A secondary tumor is a tumor that is the same type as a primary tumor (e.g., lung cancer or breast cancer) but has formed at a secondary anatomical site that is separate from the primary tumor.

In some embodiments, the mouse models are engrafted with a patient-derived xenograft (PDX). A PDX is a tumor tissue from a human or other mammal that is implanted in a mouse model of the present disclosure, for example. A PDX used herein may be obtained directly from a subject or obtained from a PDX repository. Non-limiting examples of PDX repositories include Jackson Laboratories Mouse Models of Human Cancer Database (Krupke, D M, et al., “Mouse Models of Human Cancer Database,” Nat Rev Cancer, 2008 8(6): 459-65), Dana Farber Cancer Institute Patient-Derived Tumor Xenograft Database, and Charles River Patient-Derived Xenograft Model Database. A model of pathogenic disease may comprise at least 2 (e.g., at least 3, at least 4, at least 5, at least 6, at least 7, at least 8, at least 9, or at least 10) PDXs.

A PDX may have any human tumor origin. For example, a PDX may be from bladder tumor, brain tumor, breast tumor, colon and rectal tumor, endometrial tumor, kidney tumor, leukemia, liver tumor, lung tumor, melanoma, non-Hodgkin lymphoma, ovarian cancer, pancreatic tumor, prostate tumor, sarcoma, skin cancer, testicular cancer, or thyroid tumor. Other tumor types are contemplated herein (see, e.g., cancer.gov/types).

In some embodiments, the mouse models are engrafted with human cancer cells from a human cancer cell line. Human cancer cell lines are human cancer cell cultures developed from a single cell. In some embodiments, a human cancer cell line is immortalized. Immortalized cells divide and proliferate indefinitely. Human cancer cell lines may be from any human cancer. For example, a human cancer cell line may be from a bladder tumor (HTB-9, HTB-3, CRL-2169), breast tumor (e.g., Hs.281.T, Hs 5788st, UACC-812, MCF 10A, or MDA-MB-157), brain tumor (SW 1088, U138, Daoy, LN-228), colon and rectal tumor (HT29, SW480, SW1116, Caco-2), endometrial tumor (Ishikawa), kidney tumor (Caki-1, 769-P), leukemia (MOLT-3, TALL-104, AML-193, Jurkat, Mo-B), liver tumor (e.g., SNU-182, Hs.817.T, NCI-H735, or THLE-3), lung tumor (e.g., NCI-H838, HCC827, NCI-H1666, SW 1573, ChaGo-K-1, A549, or NCI-H1555), melanoma (SK-MEL-3, A375-P, MNT-1), non-Hodgkin lymphoma (e.g., GA-10, NCI-BL2171, HH, or Toledo), ovarian tumor (SK-OV-3, PA-1, Caov-3, SW 636), pancreatic tumor (Capan-2, Panc 10.05, CFPAC-1, SQ 1990), prostate tumor (VCaP, C4-2B, LNCaP, PC-3), sarcoma (HS 822.T, SK-LMS-1, A-204), skin (TE 354.T, Hs 456.Bt,), testicular (Cates-1B, Hs 1Tes), or thyroid tumor (MDA-T120, MDA-T41, SW-579). A mouse model herein may be engrafted with human cancer cell lines from a single cell line or from multiple cell lines (e.g., at least 2, at least 3, at least 4, at least 5, at least 6, at least 7, at least 8, at least 9, or at least 10 cell lines).

Further, any of the mouse models provided herein may include a combination of human cancer cell types, optionally from a combination of sources, e.g., PDX sources and/or cell lines.

In some embodiments, the human cancer cells are genetically engineered to alter sensitivity of the cells to a pathogen (e.g., human pathogen). Genetically engineered, as provided herein, refers to human cancer cells with genomic modifications (e.g., insertions, deletions, and/or substitutions). Non-limiting examples of methods of genetically engineering human cancer cells include those based on programmable gene editing platforms such as clustered regularly interspaced short palindromic repeats/Cas nuclease (CRISPR/Cas nuclease), zinc finger nucleases (ZFNs), or transcription activator-like effector nucleases (TALENs). Other means of genetic engineering, such as recombinase-based editing, are contemplated herein.

In some embodiments, the human cancer cells may be genetically engineered to increase sensitivity to a pathogen (e.g., human pathogen) or decrease sensitivity to a pathogen, relative to a control, such as human cancer cells that have not been genetically engineered. Increasing sensitivity to a pathogen, in some embodiments, may be achieved by increasing expression and/or activity of a host cell moiety or decreasing expression of factors (e.g., proteins) that decrease or prevent pathogen entry, replication, and/or survival (e.g., antimicrobial factors). In some embodiments, human cancer cells may be genetically engineered to decrease sensitivity to a pathogen (e.g., a human pathogen). Decreasing sensitivity to a pathogen, in some embodiments, may be achieved by decreasing expression or activity of a host cell moiety or increasing expression of factors (e.g., proteins) that increase pathogen entry, replication, and/or survival (e.g., antimicrobial factors).

In some embodiments, human cancer cells are genetically engineered to express a detectable biomolecule, for example, so that the cells can be monitored in vivo or analyzed ex vivo. A detectable biomolecule, as provided herein, refers to a biomolecule in a human cancer cell that can be detected using a conventional or non-conventional assay. Non-limiting examples of detectable biomolecules include fluorescent proteins (e.g., green fluorescent protein, yellow fluorescent protein, or red fluorescent protein), antigens (e.g., hemagglutinin or human leukocyte antigen), and enzymes (e.g., beta-galactosidase or luciferase). Other detectable biomolecules are contemplated herein.

Methods for obtaining human cancer cells and PDXs include but are not limited to biopsy (e.g., hollow-needle, excisional, incisional, or brush), excision (e.g., of a tumor), and collection of a sample (e.g., blood sample) followed by a sorting step to isolate human cancer cells from other cells (e.g., human non-cancer cells, non-human cells, or cell fragments).

Engraftment

The humanized immunodeficient mouse model of pathogenic disease provide herein are engrafted with human cancer cells, for example, from a human cancer cell line or human PDX, as discussed above. Engraftment refers to the process of the human cells migrating to, and incorporating into, an existing tissue of interest in vivo.

Any tissue in a mouse may be the target tissue for engraftment of the human cancer cells. The target tissue for engraftment is the tissue to which the human cancer cells will migrate and incorporate. The target tissue for engraftment may depend on the pathogenic disease to be studied. Non-limiting examples of target tissues for engraftment of human cancer cells include lung, trachea, liver, bone marrow, brain, blood, gastrointestinal tissue, skin, stomach, small intestine, large intestine, and pancreas. Other target tissues are contemplated herein. In some embodiments, human cells will engraft in one target tissue, while in some embodiments, human cells will engraft in more than one (e.g., 2, 3, 4, 5, 6, 7, 8, 9, 10 or more) target tissue.

In some embodiments, the human cancer cells are delivered to a mouse using a single cell suspension. A single-cell suspension is a suspension of cells that lacks detectable levels of cell debris and cell aggregates. Single-cell suspensions enable separation of cells from tissues (e.g., connective tissue) and maximize the efficiency of using human cells, including, but not limited to: engraftment into a model animal (e.g., mouse model), flow cytometry, human cell culture (e.g., immortalization), and human cell labeling. Methods for preparing human cell single-cell suspensions depend on the origin of the cells (e.g., freshly isolated from human, derived from PDX, cell lines). Methods for preparing single-cell suspensions include but are not limited to dissociation (e.g., enzymatic, mechanical), purification (e.g., magnetic activated cell sorting or activated cell sorting), commercial kits (e.g., Miltenyi Biotec or StemCell), centrifugation (e.g., at >300×g), and filtering (e.g., cell strainer).

For example, the mouse may be engrafted with 1.0×10⁵-2.0×10⁷ human cancer cells. In some embodiments, the mouse is engrafted with 1.0×10⁵, 2.0×10⁵, 3.0×10⁵, 4.0×10⁵, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, or 2.0×10⁷ human cancer cells.

The human cancer cells may be delivered to a mouse via injection (e.g., tail vein, retroorbital, intravenous, intracardiac, or intraarterial) or implantation (e.g., subcutaneous, intraperitoneal, intrafemoral, intratibial, or intramuscular). Other delivery methods are contemplated herein.

Provided herein, in some embodiments, are methods of verifying engraftment of human cells. Also provided herein are methods of verifying delivery of a pathogen expressing a surface protein. These methods may depend on the source of the human cells (e.g., human cancer cells), the tissue to be engrafted, and the identity of the pathogen, for example. Any method may be used to verify engraftment of human cells (e.g., human cancer cells) and/or delivery of a pathogen. Non-limiting methods of verifying engraftment and/or delivery include: immunofluorescence imaging (e.g., when the human cells and/or pathogen comprise a fluorescent marker), flow cytometry, immunohistochemistry, monitoring expression of viral entry proteins (e.g., on human cells), monitoring expression of surface proteins (e.g., on pathogens), and measuring pathogen load.

In some embodiments, an immunodeficient model provided in the present disclosure comprises tissue (e.g., lung tissue) engrafted with human cells (e.g., PDX, human cancer cell line) expressing a host cell moiety (e.g., viral entry protein) and is infected with a pathogen expressing a surface protein (e.g., viral surface protein) that binds to a host cell moiety. In some embodiments, the pathogen is a virus. In some embodiments, the virus is a respiratory virus. In some embodiments, the respiratory virus is a coronavirus (e.g., SARS-CoV-2). In some embodiments, an immunodeficient mouse model provided in the present disclosure comprises lung tissue engrafted with human cells (e.g., PDX, human cancer cell line) expressing human ACE2 and is infected with SARS-CoV-2.

### Pathogens

The mouse models provided herein may be used to assess pathogenic infection and disease progression, to investigate the critical genes and proteins necessary for infection from new pathogens, and/or to assess new therapeutics or vaccines, for example. Pathogens are microorganisms that can cause disease in a host. Non-limiting examples of pathogens include viruses, bacteria, prions, and fungi. The mouse models provided herein are typically infected with a pathogen expressing a surface moiety that binds to a host cell moiety associated with pathogenesis on the surface of a human cancer cell that has been engrafted in the mouse models.

A host cell moiety associated with pathogenesis is a moiety located intracellularly or on the surface of a host cell that mediates pathogen (e.g., virus, bacterium, prion, or fungus) entry, replication, survival or other mechanisms that contribute to pathogenesis. In some embodiments, a host cell moiety is a pathogen entry moiety. A pathogen entry moiety is a moiety located on the surface of a host cell that mediates attachment of a pathogen (e.g., virus, bacterium, prion, or fungus) to the host cell by binding to a pathogen protein. Attachment of a virus to a host cell, for example, is mediated by virion moieties (e.g., proteins binding) to specific host viral entry moieties, non-limiting examples of which include cell surface molecules such as membrane proteins, lipids, and carbohydrate moieties present either as glycoproteins or glycolipids. Binding of a virus to a host cell leads to viral genome entry into the host cell, triggers signaling pathways, or enables the virion to be carried by host cells to a specific organ. In some embodiments, a host cell moiety associated with pathogenesis is a pathogen intracellular moiety. A pathogen intracellular moiety is located intracellularly in the host cell and mediates attachment of a pathogen (e.g., virus, bacterium, prion, or fungus) to the host cell's intracellular machinery or interferes with the host cell's physiological intracellular processes. In some embodiments, the pathogen intracellular moiety binds to endoplasmic reticulum (ER) and/or Golgi proteins in the host cell, disrupting physiological interactions in the host cell. In some embodiments, this interaction interferes with the host cell's ability to traffic viral proteins, hindering the creation of new viral particles. In some embodiments, the pathogen intracellular moiety (e.g., non-structural protein 5 (NSP5) of SARS-CoV2) binds the epigenetic regulator histone deacetylase 2 (HDAC2), inhibiting the transport of HDAC2 into the nucleus. Without wishing to be bound by theory, it is thought that the binding of a pathogen intracellular moiety to HDAC2 affects HDAC2's ability to mediate inflammation and interferon responses. As another example, a pathogen intracellular moiety may antagonize host interferon signaling by disrupting nuclear transport (e.g., ORF6 of SARS-CoV2).

Cell receptors to viruses are specific non-limiting examples of pathogen entry moieties and can be classified into two classes: adhesion receptors and entry receptors. Adhesion receptors attach the virus in a reversible manner to target cells or organs. This adhesion is not mandatory for virus entry and alone does not trigger entry. Nonetheless, it enhances infectivity by concentrating the virus in the vicinity of its entry receptors. Entry receptors trigger virus entry by endocytosis/pinocytosis or by inducing fusion/penetration, and the consequences of this binding are irreversible.

Virus attachment to a host cell may involve different binding partners. In some embodiments, a virus attaches to a host cell through a viral protein that binds to a host glycan, receptor protein, adhesion protein or peptidase. In other embodiments, a virus attaches to a host cell through a viral glycan that binds to a host cell lectin. In yet other embodiments, a virus attaches to a host cell through a viral lipid that binds a host receptor protein. Thus, surface moieties include but are not limited to pathogen surface proteins, glycans, and lipids.

In some embodiments, a pathogen is a virus that comprises a surface protein that binds to a viral entry moiety on the surface of a host cell. Viral surface proteins include but are not limited to capsid proteins and envelope proteins, for example, glycoproteins, such as Spike (S) protein, fusion proteins, hemagglutinins, and neuraminidases.

Non-limiting examples of viral entry proteins include ACE2, CDR147, TMPRSS2, sialic acid, HSPG, GM1 ganglioside, LDLR, DC-SIGN, Integrin aVb3, CD4, CCR5, CXCR4, CAR, Integrin aVb5, CD46, CD80, CD86, DSG2, VCAM-1, GD1a glycan, MHC1-alpha2, CD56, CD112, CD66a, JAM-A, CD155, CD54, CD106, PSGL-1, L-SIGN, TIM-1, Hsp70, HBGA, Hsp70, alpha-dystroglycan, Transferrin-receptor, ICAM-1, SLAM, GLUT-1, Neuropilin-1, CD81, SR-B1, CD21, and MHC-II. Other viral surface proteins are contemplated herein.

Viruses

The viruses most deadly to humans, any of which may be used to infect the mouse models provided herein, include Marburg virus, Ebola virus, Rabies, HIV, Smallpox, Hantavirus, Influenza, Dengue, Rotavirus, severe acute respiratory syndrome coronavirus (SARS-CoV), severe acute respiratory syndrome coronavirus 2 (SARS-CoV-2), and Middle East respiratory syndrome-related coronavirus (MERS-CoV).

In some embodiment, the virus is a respiratory virus. Non-limiting examples of respiratory viruses include rhinoviruses and enteroviruses (Picornaviridae), influenza viruses (Orthomyxoviridae), parainfluenza, metapneumoviruses and respiratory syncytial viruses (Paramyxoviridae), coronaviruses (Coronaviridae), and several adenoviruses. Other respiratory viruses are contemplated herein.

In some embodiments, a virus is a Flaviviridae virus. The Flaviviridae are a family of positive, single-stranded, enveloped RNA viruses. They are found in arthropods, (primarily ticks and mosquitoes), and can occasionally infect humans. Members of this family belong to a single genus, Flavivirus, and cause widespread morbidity and mortality throughout the world. Some of the mosquitoes-transmitted viruses include: Yellow Fever, Dengue Fever, Japanese encephalitis, West Nile viruses, and Zika virus. Other Flaviviruses are transmitted by ticks and are responsible of encephalitis and hemorrhagic diseases: Tick-borne Encephalitis (TBE), Kyasanur Forest Disease (KFD) and Alkhurma disease, and Omsk hemorrhagic fever.

In some embodiments, a virus is a Togaviradae virus. Togaviridae is a family of small, enveloped viruses with single-stranded positive-sense RNA genomes of 10-12 kb. Within the family, the Alphavirus genus includes a large number of species that are mostly mosquito-borne and pathogenic in their vertebrate hosts. Many are important human and veterinary pathogens (e.g., chikungunya virus, eastern equine encephalitis virus). Before April 2019 the family also contained the genus Rubivirus that has now been moved to the family Matonaviridae. The present disclosure contemplates viruses of the Togaviridae family as well as the Matonaviridae family.

In some embodiments, a virus is a Bunyaviriade virus. Bunyavirales is an order of single-strand, spherical, enveloped RNA viruses (formerly the Bunyaviridae family). The virus families in the Bunyavirales order that cause viral hemorrhagic fevers include Phenuiviridae, Arenaviridae, Nairoviridae, and Hantaviridae. Distribution of these viruses is determined by the distribution of the vector and host species. In some embodiments, the virus is LaCrosse virus. In some embodiments, the respiratory virus is a coronavirus. Non-limiting examples of coronaviruses include alphacoronaviruses and betacoronaviruses. In some embodiments, the virus is a betacoronavirus. Non-limiting examples of coronaviruses include SARS-CoV, SARS-CoV-2, MERS-CoV, human coronavirus HKU1, 229E, NL63I OC43 and human coronavirus OC43.

In some embodiments, the virus is SARS-CoV-2, which causes COVID-19. In such embodiments, the mouse models provided herein comprise human cancer cells that express ACE2, TMPRSS2, and/or CD147, which mediates viral attachment by binding to S protein of SARS-COV-2. Recent reports demonstrate that while angiotensin I converting enzyme 2 (ACE2) is the main receptor for the S protein of SARS-CoV-2 thus mediating viral attachment to target cells, several other proteins are important for successful infection. The transmembrane protease, serine 2 (TMPRSS2), for example, cleaves protein S at the S1/S2 and the S2 sites, allowing fusion of viral and cellular membranes. SARS-CoV-2 S protein contains four redundant FURIN cut sites making FURIN another potential protease involved in priming for host cell entry. Additionally, CD147 (also known as Basigin (BSG) or extracellular matrix metalloproteinase inducer (EMMPRIN)) may bind spike protein of SARS-CoV-2 and possibly be involved in host cell invasion. It is not yet clear whether CD147-mediated viral entry also requires TMPRSS2, whether ACE2 and CD147 function as part of a single pathway or in parallel, or what other key factors are involved. Given the variable infection rates and mortality rates, it is possible there are several unknown modulators of infectivity or disease severity.

Bacteria

Non-limiting examples of bacteria that may be used to infect the mouse models provided herein include Legionella pneumophila, Listeria monocytogenes, Campylobacter jejuni, Staphylococcus aureus, Escherichia coli, Borrelia burgdorferi, Helicobacter pylori, Ehrlichia chaffeensis, Clostridium difficile, Vibrio cholerae, Salmonella enterica, Bartonella henselae, Streptococcus pyogenes (Group A Strep), multiple drug resistant S. aureus (e.g. MRSA), Chlamydia pneumoniae, Clostridium botulinum, Vibrio vulrificus, Parachlamydia, Corynebacterium amycolatum, Klebsiella pneumoniae, Linezolid-resistant enterococci (E. faecalis and E. faecium), and multiple drug resistant Acinetobacter baumannii. Other bacteria are contemplated herein.

Prions

A prion is not a pathogen but rather a type of protein that can trigger normal proteins in the brain to fold abnormally. The most common form of prion disease that affects humans is Creutzfeldt-Jakob disease (CJD). Non-limiting examples of prions that may be administered to the mouse models provided herein include PrP^{c}, Pp^{res}, and PRP^{Sc}. Other prions are contemplated herein.

Fungi

A fungus is any member of the group of eukaryotic organisms that includes microorganisms such as yeasts and molds. Non-limiting examples of fungi that may be used to infect the mouse models provided herein include Candida albicans, Cryptococcus neoformans, Aspergillus flavus, and Candida tropicalis. Other fungi are contemplated herein.

Methods for delivering a pathogen to a mouse include but are not limited to inhalation (e.g., nasal or tracheal), injection (e.g., intravenous, cranial, hepatic artery, or peritoneal injection), and ingestion (e.g., oral or rectal).

### Collections of Immunodeficient Mice

Provided herein, in some embodiments, is a collection of humanized immunodeficient models (e.g., mice) of pathogenic disease. A collection of mouse models refers to at least 2 (e.g., at least 3, 4, 5, 10, 15, 20, 25, 30, 35, 40, 45, 50, or more) mouse models.

In some embodiments, each mouse in a collection comprises tissues engrafted with human cells from a different cancer source. Any tissue in an immunodeficient mouse of a collection may be engrafted with human cells including, but not limited to lung tissue, tracheal tissue, nasal cavity tissue, liver tissue, kidney tissue, bone tissue, brain tissue, small intestine tissue, and large intestine tissue. In some embodiments, each immunodeficient mouse in a collection comprises tissues engrafted with human cells from a primary tumor, a secondary tumor, a PDX, and/or a human cancer cell line.

Human cells (e.g., human cancer cells) in immunodeficient mouse models provided herein may express host cell moieties. In some embodiments, human cells in each mouse in a collection of immunodeficient mouse models of pathogenic disease express a single host cell moiety. In some embodiments, human cells in each mouse in a collection of immunodeficient mouse models of pathogenic disease express a combination of host cell moieties. In some embodiments, a single mouse in a collection of immunodeficient mouse models of pathogenic disease expresses a host cell moiety. In some embodiments, multiple (e.g., 2 or more) mice in a collection of immunodeficient mouse models of pathogenic disease express a host cell moiety. Thus, in some embodiments, each immunodeficient mouse in a collection will have variable expression of a host cell moiety, with some mice having higher (e.g., relative to a control) expression of a host cell moiety, some mice having lower (e.g., relative to a control) expression of a host cell moiety, and some mice having comparable (e.g., relative to a control) expression of a host cell moiety. A control is a mouse that is not engrafted with human cells or a mouse engrafted with human cells that are not susceptible to a pathogen (e.g., do not express detectable levels of a host cell moiety).

Provided herein, in some embodiments, are methods of using a collection of immunodeficient models (e.g., mouse models) of pathogenic disease. Non-limiting examples of methods of use include studying: expression of individual factors (e.g., genes or host cell moieties) in pathogenic disease establishment, progression of pathogenic disease, and methods of treatment.

In some embodiments, provided herein are methods of using a collection of immunodeficient models (e.g., mouse models) of pathogenic disease to study expression of individual factors (e.g., genes, proteins, or RNAs) in pathogenic disease establishment. Such models may be used, for example, to elucidate critical genes and proteins necessary for infection of new pathogens. Methods of studying individual factors may depend on the type (e.g., gene, protein, or RNA) of individual factor. Any method may be used to study individual factors in pathogenic disease establishment including, but not limited to DNA microarray analysis, reverse transcription PCR (RT-PCR), Western blot, ELISA, RNA-sequencing (RNA-Seq), flow cytometry, and co-immunoprecipitation.

In some embodiments, provided herein are methods of using a collection of immunodeficient models (e.g., mouse models) of pathogenic disease to study progression of pathogenic disease. Progression of pathogenic disease may depend on the identity of the pathogen and the symptoms of pathogenic disease. Non-limiting methods for monitoring progression of pathogenic disease include measuring expression of biomarkers (e.g., viral entry or proteins), monitoring symptoms (e.g., fever, weight loss, lethargy, or difficulty breathing), and monitoring antibody production.

In some embodiments, provided herein are methods of using a collection of immunodeficient models (e.g., mouse models) of pathogenic disease to study methods of treating pathogenic disease. Methods of treating pathogenic disease may depend on the identity of the pathogen and include but are not limited to administering: one or more therapeutic agents and palliative care. Therapeutic agents are discussed in greater detail below. Palliative care, as used herein, refers to interventions to treat the symptoms (e.g., fever, weight loss, lethargy, difficulty or breathing) of a pathogenic disease.

### Methods of Use

A humanized, immunodeficient mouse model of pathogenic disease provided herein may be used for any number of applications. For example, a humanized, immunodeficient model (e.g., immunodeficient mouse model) may be used to assess pathogenic behavior, assess pathogenic impact on the human immune system, and/or test how a candidate prophylactic agent or a candidate therapeutic agent affects the human immune system in addition to other organs or systems including, but not limited to, the cardio-vascular system, renal function, hepatic function, and the nervous system following pathogenic infection.

Assessment of Pathogenic Behavior

Immunodeficient models (e.g., mouse models) provided herein may be used to assess pathogenic behavior. Pathogenic behavior refers to the changes that occur in a host (e.g., immunodeficient mouse model) due to pathogenic infection. Non-limiting examples of pathogenic behavior include production of biomarkers of pathogenic infection, spread of pathogenic infection to multiple tissues, production of symptoms of pathogenic infection, and production of critical genes and/or proteins for pathogenic infection of a novel pathogen.

Provided herein, in some embodiments, are methods of assessing production of biomarkers of pathogenic infection. Biomarkers of pathogenic infection may vary depending on the pathogen and include but are not limited to lipopolysaccharide (LPS), cholera toxin, pathogen genomic material, and agr operon autoinducer peptide. Any method may be used to assess biomarker production. Non-limiting examples of methods used in the art include detection of biomarkers in clinical samples (e.g., blood, urine, throat swab, or cerebrospinal fluid), and RT-PCR, ELISA, and Western blot of pathogenic biomarkers.

Provided herein, in some embodiments, are methods of assessing spread of pathogenic infection to multiple tissues in a host (e.g., immunodeficient mouse model). Multiple tissues may be multiple examples of the same type of tissue (e.g., lung tissue) or multiple different tissue types (e.g., lung tissue, blood, liver, or brain). Spread of pathogenic infection (e.g., from the primary tissue(s) that a pathogen infects) may depend on the identity of the pathogen and any underlying diseases or disorders in the host. Any method may be used to assess spread of pathogenic infection including, but not limited to: obtaining multiple tissue samples and (i) verifying (e.g., immunohistochemistry) presence of pathogens and/or (ii) measuring pathogen surface protein expression; and performing in vivo imaging system (IVIS) (e.g., with a fluorescent marker).

Provided herein, in some embodiments, are methods of assessing production of symptoms of pathogenic infection. The symptoms of pathogenic infection may depend on the identity of the pathogen and the host (e.g., humanized immunodeficient mouse model). It is desirable that symptoms of pathogenic infection in models provided in the present disclosure mimic symptoms of pathogenic infection in humans. Non-limiting examples of symptoms of pathogenic infection include fever, lethargy, weight loss, diarrhea, muscle aches, coughing, difficulty breathing, vomiting, seizures, ataxia, change in blood pressure, proteinuria, hematuria, loss of fur, edema, erythema, dermatitis, and dehydration. Any method may be used to assess production of symptoms of pathogenic infection. Non-limiting examples of methods used to assess production of symptoms of pathogenic infection include measuring temperature, monitoring sleep/wake cycles, monitoring activity, measuring weight, assessing solid and liquid excrement, monitoring breathing and assessing lung function, monitoring blood pressure and cardiac function, measuring protein or blood presences in the urine, measuring cytokine levels in the blood, evaluating changes in skin or mucous membrane thickness, measuring ion concentration in blood, and any disease activity index (DAI) evaluation.

Provided herein, in some embodiments, are methods of determining critical genes and/or proteins necessary for infection of a pathogen, including a novel pathogen. For example, a panel of genetically diverse PDXs or PDX-derived cell lines may be infected by the new pathogen. By assessing the resulting viral loads in vitro, the genetic similarities between the PDXs that enable infection and/or differences between those that do not, may be investigated to determine the critical genes and proteins necessary for infection, such as proteins critical for viral entry. In some embodiments, viral loads resulting from infection are compared to a reference value (e.g., uninfected control cells or cell line). In some embodiments, genetic mapping is used to determine which gene or genes are involved in viral entry. Examples of viral entry proteins include, but are not limited to, ACE2, CDR147, TMPRSS2, sialic acid, HSPG, GM1 ganglioside, LDLR, DC-SIGN, Integrin aVb3, CD4, CCR5, CXCR4, CAR, Integrin aVb5, CD46, CD80, CD86, DSG2, VCAM-1, GDIa glycan, MIHIC1-alpha2, CD56, CD112, CD66a, JAM-A, CD155, CD54, CD106, PSGL-1, L-SIGN, TIM-1, Hsp70, HBGA, Hsp70, alpha-dystroglycan, Transferrin-receptor, ICAM-1, SLAM, GLUT-1, Neuropilin-1, CD81, SR-B1, CD21, and MHC-II. After identifying critical genes relating to the infectivity or transmissibility of the new pathogen, the most effective PDX or PDX-derived cell lines can then be used to build a model for studying the new pathogen and helping to find a new therapeutic or vaccine, as described herein. In a further embodiment, the critical genes identified may be used to genetically modify and produce animal models (e.g., mice) that express the critical genes. In this way, additional in vivo studies of pathogen replication and disease may be performed.

Assessment of Pathogenic Impact on Human Immune System

Humanized immunodeficient mouse models provided herein may be used to assess pathogenic impact on the human immune system. Non-limiting examples of pathogenic impact on the human immune system include modulated human immune cell production, production of antibodies that bind pathogens, and cytokine release.

Provided herein, in some embodiments, are methods of assessing modulated human immune cell production. Modulated human immune cell production may be increased human immune cell production (e.g., compared to a control) or decreased human immune cell production (e.g., compared to a control). A control may be a humanized immunodeficient model (e.g., mouse model) not infected with a pathogen. The production of any human immune cell in a humanized immunodeficient model (e.g., mouse models) may be modulated by pathogenic infection. Non-limiting examples of human immune cells whose production may be modulated by pathogenic infection include: hematopoietic stem cells (e.g., surface marker CD34+), T-cells (e.g., surface markers CD3+, CD4+, CD8+), B cells (e.g., surface marker CD19+, CD20+), natural killer cells, plasma cells, immunoglobulins, neutrophils, monocytes, dendritic cells, and cytokines (e.g., IL-2, IL-4, or IL-6). Any method of measuring human immune cell production may be used to assess modulated human immune cell production. Non-limiting examples of methods of measuring human immune cell production include flow cytometry, fluorescence activated cell sorting (FACS), RT-PCR of human immune cell surface markers and cytokines, and ELISA.

Provided herein, in some embodiments, are methods of assessing production of antibodies by humanized immunodeficient models (e.g., mouse models) provided by the present disclosure. Antibodies are produced by human plasma cells to bind specific antigens (e.g., pathogen) and initiate a complex chain of events in the human immune system to destroy the antigen (e.g., pathogen). Assessing production of antibodies may be by any method including, but not limited to: ELISA, Western blot, and RT-PCR.

Assessment of Prophylactic and Therapeutic Agents

Immunodeficient models (e.g., mouse models) provided in the present disclosure may be used to assess prophylactic agents and therapeutic agents for preventing or treating pathogenic infection. A prophylactic agent is a substance (e.g., drug or protein) that prevents or reduces risk of pathogenic infection or prevents or reduces the risk of the development of disease following pathogenic infection. A therapeutic agent is a substance (e.g., drug or protein) that treats pathogenic infection. Therapeutic agents include palliative agents, which are substances (e.g., drug or protein) that ameliorates one or more symptoms of a pathogenic infection.

With respect to prevention of a pathogenic (e.g., viral) infection, it should be understood that a prophylactically effective amount of an agent need not entirely eradicate the pathogen (e.g., virus) but should prevent the pathogenic particles present in the subject from causing symptoms of a disease (e.g., high fever, difficulty breathing, or nausea). In some embodiments, a prophylactically effective amount of an agent reduces the pathogenic particle population present in the subject by at least 30%, at least 40%, at least 50%, at least 60%, at least 70%, at least 80%, or at least 90%. Likewise, with respect to treatment of a pathogenic infection, it should be understood that a therapeutically effective amount of an agent need not cure a disease associated with a pathogenic infection or entirely eradicate the pathogenic particles but should alleviate at least one symptom of the disease and reduce the pathogenic particle population present in the subject by at least 30%, at least 40%, at least 50%, at least 60%, at least 70%, at least 80%, or at least 90%. It should be understood that a therapeutic agent (e.g., a palliative agent) need not impact the pathogenic particle population present in the subject at all but should alleviate at least one symptom of the disease and thus potentially mitigate the short- or long-term systemic impact of the pathogenic infection.

A prophylactic agent and/or a therapeutic agent may be delivered by any method. Non-limiting examples of methods of delivering a prophylactic agent and/or a therapeutic agent include: inhalation (e.g., nasal or tracheal), injection (e.g., intravenous, intraarterial, intramuscular, or intracranial), and ingestion (e.g., tablet or liquid).

In some embodiments, the candidate agent is convalescent human serum convalescent human serum is serum comprising antibodies from a human who has been infected with the pathogen (e.g., virus).

In some embodiments, the candidate agent is a human vaccine. Human vaccines against a pathogen (e.g., virus) may contain activated (live) pathogen (e.g., virus), inactivated (killed) pathogen, nucleic acids (e.g., DNA, RNA) that block transcription or translation of pathogenic proteins, recombinant pathogenic (e.g., viral) protein, and licensed vectors.

In some embodiments, the candidate agent is an antimicrobial agent, such as an antibacterial agent and/or an antiviral agent, including but not limited to: lopinavir, ritonavir, remdesivir, favipiravir, ivermectin, recombinant human ACE2, umifenovir, recombinant interferon, chloroquine, and hydroxychloroquine.

In some embodiments, a candidate agent is an analgesic, anti-pyretic, anti-inflammatory drug, or an immunosuppressive including but not limited to NSAIDs, steroids, diuretics, statins, and beta-blockers.

Combinations of any of the prophylactic agents and/or therapeutic agents provided herein may also be administered to an immunodeficient model (e.g., mouse) infected with a pathogen. In some embodiments, an immunodeficient model infected with a pathogen is administered 1 or more, 2 or more, 3 or more, 4 or more, 5 or more, 6 or more, 7 or more, 8 or more, 9 or more, or 10 or more prophylactic agents. In some embodiments, an immunodeficient model (e.g., mouse) infected with a pathogen is administered 1 or more, 2 or more, 3 or more, 4 or more, 5 or more, 6 or more, 7 or more, 8 or more, 9 or more, or 10 or more therapeutic (e.g., palliative) agents. In some embodiments, an immunodeficient model (e.g., mouse) infected with a pathogen is administered 1 or more, 2 or more, 3 or more, 4 or more, 5 or more, 6 or more, 7 or more, 8 or more, 9 or more, or 10 or more prophylactic and therapeutic agents.

Any effective amount of a prophylactic agent and/or a therapeutic agent may be administered to a subject (e.g., immunodeficient model or human patient). An effective amount is a dose (e.g., mg, mg/mL, or mg/kg) that prevents or reduces the risk of developing a pathogenic (e.g., viral) infection and/or treats a pathogenic infection (e.g., eradicates the pathogen and/or mitigates the side effects from the pathogenic (e.g., viral) infection). Any dosage regimen may be used to administer a prophylactic agent and/or a therapeutic agent to a subject. Non-limiting examples of dosage regimens include 1 dose daily-24 doses daily, 1 dose weekly-7 doses weekly, 1 dose monthly-30 doses monthly, 1 dose every 2 months-1 dose every year, 1 dose yearly-1 dose every 10 years or more.

The efficacy of a prophylactic agent and/or a therapeutic agent may be assessed by any method. Non-limiting examples of assessing the efficacy of a prophylactic agent and/or a therapeutic agent include monitoring: pathogen (e.g., viral) load, production of antibodies, prevention of infection by the pathogen, development of disease caused by the pathogen, and systemic function of an immunodeficient mouse infected with the pathogen.

Provided herein, in some embodiments, are methods of measuring pathogen (e.g., viral) load. Pathogen load refers to the amount of pathogen present in an immunodeficient model of pathogenic disease. Pathogen load may be measured by any method (see, e.g., Lazcka, et al., “Pathogen detection: a perspective of traditional methods and biosensors,” Biosensors and Bioelectronics, 2007, 7: 1205-17) including, but are not limited to: polymerase chain reaction to detect a pathogenic protein, pathogen culture, colony counting, and counting of detection of antigen-antibody interactions.

Also provided herein, in some embodiments, are methods of monitoring production of antibodies against a pathogen (e.g., virus) in an immunodeficient model of pathogenic disease. Antibody productions may be monitored by any method including, but not limited to: ELISA, flow cytometry, Western blot, and enzyme-linked immunospot assay.

Further provided herein, in some embodiments, are methods of monitoring prevention or treatment of infection and disease development by a pathogen in an immunodeficient model. Prevention or treatment of disease and disease development may be monitored by any method and may depend on the pathogen. Non-limiting examples of monitoring prevention or treatment of pathogenic infection and disease development include: measuring temperature, measuring body weight, monitoring movement, assessing sleep/wake cycles, performing DAI scoring, and measuring pathogen load.

Provided herein, in some embodiments, are methods of monitoring systemic function of an immunodeficient model infected with a pathogen. Systemic function refers to productivity of an organ system in an immunodeficient model infected with a pathogen. Any organ system in a model (e.g., respiratory, cardiac, digestive, renal, endocrine, or nervous) may be monitored in methods provided herein. Non-limiting examples of monitoring systemic function include: measuring respiratory function (e.g., spirometry, lung capacity and airway resistance, diffusing capacity, blood gas analysis, or cardiopulmonary exercise testing), cardiac function (e.g., cardiac catheterization, pulsed Doppler measures of blood pressure, Doppler blood flow studies, peripheral vessel stiffness and flow velocity), kidney/renal function (proteinuria, creatinine levels, BUN), liver function (albumin, ALT, AST, bilirubin), and neural function (e.g., patch clamp, functional MRI, gait analysis, or balance tests).

### Additional Embodiments

Some aspects of the present disclosure provide an immunodeficient mouse engrafted with human cancer cells comprising a host cell moiety associated with pathogenesis, wherein the mouse is infected with a pathogen comprising a surface moiety that binds to the host cell moiety. The human cancer cells, in some embodiments, are from a human cancer cell line or a patient-derived xenograft (PDX).

In some embodiments, the human cancer cells are genetically engineered to alter sensitivity of the cells to a human pathogen, for example, to increase or decrease sensitivity of the cell to infection.

In some embodiments, the human cancer cells are genetically engineered to express a detectable biomolecule, optionally a fluorescent or bioluminescent protein. Such detectable marking enables in vivo and ex vivo assessment of certain cells and molecules affected by pathogen infection, for example.

In some embodiments, lung tissue, liver tissue, gastrointestinal tissue, brain tissue, skin tissue, and/or bone marrow of the mouse is engrafted with the human cancer cells. Other tissues and organs are contemplated herein.

In some embodiments, an immunodeficient mouse has a non-obese diabetic (NOD) genotype. In some embodiments, a mouse comprises a null mutation in a murine Prkdc gene. In some embodiments, the mouse comprises a null mutation in a murine Il2rg gene. In some embodiments, the mouse has a NOD scid gamma (NSG™) genotype (i.e., NOD.Cg-Prkdc^{scid}Il2rg^{tm1Wj1}/SzJ).

In some embodiments, an immunodeficient mouse is deficient in murine major histocompatibility complex (MHC) Class I and murine MHC Class II expression. In some embodiments, an immunodeficient mouse comprises a null mutation in a murine H2-Ab1 gene, a null mutation in a murine H2-K1 gene, and a null mutation in a murine H2-D1 gene. For example, an immunodeficient mouse may have a NOD.Cg-Prkdc^{scid}H2-Ab1^{em1Mvw} H2-K1^{tm1Bpe} H2-D1^{tm1Bpe} Il2^{tm1Wj1}/SzJ genotype.

In some embodiments, a human PDX is from a bladder tumor, brain tumor, breast tumor, colon and rectal tumor, endometrial tumor, kidney tumor, leukemia, liver tumor, lung tumor, melanoma, non-Hodgkin lymphoma, ovarian tumor, pancreatic tumor, prostate tumor, sarcoma, skin tumor, testicular tumor, or thyroid tumor. Other tumor tissues are contemplated herein.

In some embodiments, an immunodeficient mouse is engrafted with human peripheral blood mononuclear cells (PMBCs) or human hematopoetic stem cells (HSCs). In some embodiments, the PMBCs or HSCs are HLA-matched to the PDX.

In some embodiments, a host cell moiety is selected from membrane proteins, lipids, and carbohydrate moieties, optionally present either on glycoproteins or glycolipids.

In some embodiments, a surface protein is selected from proteins, glycans, and lipids.

In some embodiments, a pathogen is selected from bacteria, viruses, prions, and fungi.

In some embodiments, a virus is a respiratory virus. For example, a respiratory virus may be selected from influenza virus, respiratory syncytial virus, parainfluenza viruses, metapneumovirus, rhinovirus, coronaviruses, adenoviruses, and bocaviruses. In some embodiments, a respiratory virus is a coronavirus, for example, an alphacoronavirus or a beta coronavirus. In some embodiments, a coronavirus is selected from 229E, NL631 OC43, HKU1, MERS-CoV, SARS-CoV, and SARS-CoV-2. In some embodiments, a coronavirus is SARS-CoV-2.

In some embodiments, a virus is a Flaviviridae virus (e.g., yellow fever virus, West Nile virus, Dengue virus, Zika virus, Powassan virus).

In some embodiments, a virus is a Togaviradae virus (e.g., Chikungunya virus).

In some embodiments, a virus is a Bunyaviriade virus (e.g., LaCrosse virus).

In some embodiments, a surface protein is a surface glycoprotein, optionally a spike (S) protein. Other surface proteins, including other glycoproteins, are contemplated herein.

In some embodiments, a host cell moiety is a protein selected from ACE2, CD147, and TMPRSS2. It should be understood that the host cell moiety depends on the nature of the surface protein, which depends on the type of pathogen, e.g., virus.

Other aspects of the present disclosure provide an immunodeficient mouse comprising lung tissue engrafted with human cancer cells from a patient-derived xenograft or cell line, wherein the human cancer cells comprise a host cell moiety, optionally a viral entry moiety, and the mouse is infected with a pathogen comprising a surface moiety that binds to the host cell moiety.

Some aspects of the present disclosure provide an immunodeficient mouse comprising lung tissue engrafted with human cancer cells from a patient-derived xenograft or cell line, wherein the human cancer cells comprise human a viral entry moiety and the mouse is infected with a blood-borne virus comprising a surface moiety that binds to the viral entry moiety.

Yet other aspects of the present disclosure provide an immunodeficient mouse comprising lung tissue engrafted with human cancer cells from a patient-derived xenograft or cell line, wherein the human cancer cells comprise human a viral entry moiety and the mouse is infected with a respiratory virus, optionally a coronavirus, comprising a surface moiety that binds to the viral entry moiety.

Still other aspects of the present disclosure provide an immunodeficient mouse comprising lung tissue engrafted with human cancer cells from a patient-derived xenograft or cell line, wherein the human cancer cells comprise human ACE2 and the mouse is infected with SARS-CoV-2. In some embodiments, a mouse has a non-obese diabetic (NOD) scid gamma genotype. In some embodiments, a mouse has a NOD.Cg-Prkdc^{scid} H2-Ab1^{em1Mvw} H2-K1^{tm1Bpe}H2-D1^{tm1Bpe} Il2rg^{tm1Wj1}/SzJ genotype.

Some aspects of the present disclosure provide a method comprising delivering to an immunodeficient mouse human cancer cells comprising a host cell moiety and delivering to the mouse a pathogen comprising a surface moiety that binds to the host cell moiety.

Other aspects of the present disclosure provide a method comprising delivering to an immunodeficient mouse human cancer cells comprising a host cell moiety, wherein the mouse is infected with a pathogen comprising a surface moiety that binds to the host cell moiety.

Yet other aspects of the present disclosure provide a method comprising delivering to an immunodeficient mouse a pathogen comprising a surface moiety that binds to a host cell moiety, wherein the mouse is engrafted with human cancer cells comprising the host cell moiety.

Still other aspects of the present disclosure provide a method comprising: preparing a single cell suspension of human patient-derived xenograft (PDX) cells obtained from a mouse, wherein the human PDX cells comprise human cancer cells comprising a host cell moiety; delivering to an immunodeficient mouse a sample of the single cell suspension; and delivering to the immunodeficient mouse a pathogen comprising a surface moiety that binds to the host cell moiety.

In some embodiments, a single cell suspension has been purified to remove mouse cells.

In some embodiments, a sample of the single cell suspension is delivered by tail vein injection, cardiac injection, caudal artery injection, cranial injection, hepatic artery injection, femoral injection, peritoneal injection, or tibial injection.

In some embodiments, the method further comprises delivering to the mouse a therapeutic agent (e.g., a palliative agent) or a prophylactic agent.

In some embodiments, the method further comprises assessing toxicity of the agent. In some embodiments, the method further comprises assessing efficacy of the agent for treating or preventing infection by the virus and/or development of a disease caused by the pathogen.

In some embodiments, the method further comprises assessing one or more of the following: viral load in the mouse; viral titer in the mouse; respiratory function and/or cardiac function of the mouse; the human immune cell-mediated response to the virus; and a biomarker of viral disease progression.

Some aspects of the present disclosure provide a collection of immunodeficient mice, wherein each mouse of the collection comprises mouse tissue engrafted with human patient-derived xenograft (PDX) cells from a different tumor, and the human PDX cells of each mouse of the collection express a combination of host cell moieties, wherein expression levels of the host cell moieties vary among mice of the collection. In some embodiments, each mouse is infected with a pathogen.

Some aspects of the present disclosure provide a method comprising infecting multiple single-cell suspensions with a pathogen, wherein each of the single-cell suspensions comprises human cells of a different xenograft; measuring viral load for each of the single-cell suspensions; and identifying a single-cell suspension having a viral load greater than a reference value.

In some embodiments, the method further comprises delivering to an immunodeficient mouse a sample of the single cell suspension having a viral load greater than a reference value.

In some embodiments, the method further comprises assessing genetic differences among the human cells of the multiple single-cell suspensions. In some embodiments, the method further comprises genetically mapping genes of the human cells necessary for pathogenic infection. In some embodiments, the method further comprises identifying at least one pathogen entry moiety used by the pathogen for entry into the human cells.

In some embodiments, the method further comprises delivering to the immunodeficient mouse the pathogen. In some embodiments, the method further comprises delivering to the immunodeficient mouse a therapeutic agent or a prophylactic agent.

The present disclosure also contemplates the embodiments encompassed by the following numbered paragraphs:

1. An immunodeficient mouse engrafted with human cancer cells comprising a host cell moiety associated with pathogenesis, wherein the mouse is infected with a pathogen comprising a surface moiety that binds to the host cell moiety.

2. The immunodeficient mouse of paragraph 1, wherein the human cancer cells are from a human cancer cell line or a patient-derived xenograft (PDX).

3. The immunodeficient mouse of paragraph 1 or 2, wherein the human cancer cells are genetically engineered to alter sensitivity of the cells to a human pathogen.

4. The immunodeficient mouse of any one of the preceding paragraphs, wherein the human cancer cells are genetically engineered to express a detectable biomolecule, optionally a fluorescent or bioluminescent protein.

5. The immunodeficient mouse of any one of the preceding paragraphs, wherein lung tissue, liver tissue, gastrointestinal tissue, brain tissue, skin tissue, and/or bone marrow of the mouse is engrafted with the human cancer cells.

6. The immunodeficient mouse of any one of the preceding paragraphs, wherein the mouse has a non-obese diabetic (NOD) genotype.

7. The immunodeficient mouse of any one of the preceding paragraphs, wherein the mouse comprises a null mutation in a murine Prkdc gene.

8. The immunodeficient mouse of any one of the preceding paragraphs, wherein the mouse comprises a null mutation in a murine Il2rg gene.

9. The immunodeficient mouse of any one of the preceding paragraphs, wherein the mouse has a NOD scid gamma genotype.

10. The immunodeficient mouse of any one of the preceding paragraphs, wherein the mouse is deficient in murine MHC Class I and murine MHC Class II expression.

11. The immunodeficient mouse of paragraph 10, wherein the mouse comprises a null mutation in a murine H2-Ab1 gene, a null mutation in a murine H2-K1 gene, and a null mutation in a murine H2-D1 gene.

12. The immunodeficient mouse of paragraph 11, wherein the mouse has a NOD.Cg-Prkdcscid H2-Ab1em1Mvw H2-K1tm1Bpe H2-D1tm1Bpe Il2rgtm1Wj1/SzJ genotype.

13. The immunodeficient mouse of any one of paragraphs 2-12, wherein the human PDX is from a bladder tumor, brain tumor, breast tumor, colon and rectal tumor, endometrial tumor, kidney tumor, leukemia, liver tumor, lung tumor, melanoma, non-Hodgkin lymphoma, ovarian tumor, pancreatic tumor, prostate tumor, sarcoma, skin tumor, testicular tumor or thyroid tumor.

14. The immunodeficient mouse of any one of the preceding paragraphs, wherein the mouse is engrafted with human peripheral blood mononuclear cells (PMBCs) or human hematopoietic stem cells (HSCs), optionally wherein the PMBCs or HSCs are HLA-matched to the human PDX.

15. The immunodeficient mouse of any one of the preceding paragraphs, wherein the host cell moiety is selected from membrane proteins, lipids, and carbohydrate moieties, optionally present either on glycoproteins or glycolipids.

16. The immunodeficient mouse of any one of the preceding paragraphs, wherein the surface protein is selected from proteins, glycans, and lipids.

17. The immunodeficient mouse of any one of the preceding paragraphs, wherein the pathogen is selected from bacteria, viruses, prions, and fungi.

18. The immunodeficient mouse of paragraph 17, wherein the pathogen is a virus.

19. The immunodeficient mouse of paragraph 18, wherein the virus is a respiratory virus.

20. The immunodeficient mouse of paragraph 19, wherein the respiratory virus is selected from influenza viruses, respiratory syncytial viruses, parainfluenza viruses, adenoviruses, and coronaviruses.

21. The immunodeficient mouse of paragraph 20, wherein the respiratory virus is a coronavirus, optionally an alphacoronavirus or a beta coronavirus.

22. The immunodeficient mouse of paragraph 21, wherein the coronavirus is selected from 229E, NL631 OC43, HKU1, MERS-CoV, SARS-CoV, and SARS-CoV-2.

23. The immunodeficient mouse of paragraph 22, wherein the coronavirus is SARS-CoV-2.

24. The immunodeficient mouse of paragraph 23, wherein the surface protein is a surface glycoprotein, optionally a spike (S) protein.

25. The immunodeficient mouse of paragraph 23 or 24, wherein the pathogen entry moiety is a protein selected from ACE2, CD147, and TMPRSS2.

26. An immunodeficient mouse comprising lung tissue engrafted with human cells from a patient-derived xenograft tumor or cell line, wherein the human cells comprise a pathogen entry moiety, optionally a viral entry moiety, and the mouse is infected with a pathogen comprising a surface moiety that binds to the pathogen entry moiety.

27. An immunodeficient mouse comprising lung tissue engrafted with human cells from a patient-derived xenograft tumor or cell line, wherein the human cells comprise human a viral entry moiety and the mouse is infected with a respiratory virus, optionally a coronavirus, comprising a surface moiety that binds to the viral entry moiety.

28. An immunodeficient mouse comprising lung tissue engrafted with human cells from a patient-derived xenograft tumor or cell line, wherein the human cells comprise human ACE2 and the mouse is infected with SARS-CoV-2.

29. The immunodeficient mouse of any one of paragraphs 26-28, wherein the mouse has a non-obese diabetic (NOD) scid gamma genotype.

30. The mouse of paragraph 29, wherein the mouse has a NOD.Cg-Prkdcscid H2-Ab1em1Mvw H2-K1tm1Bpe H2-D1tm1Bpe Il2rgtm1Wj1/SzJ genotype.

31. A method comprising delivering to an immunodeficient mouse human cancer cells comprising a pathogen entry moiety and delivering to the mouse a pathogen comprising a surface moiety that binds to the pathogen entry moiety.

32. A method comprising delivering to an immunodeficient mouse human cancer cells comprising a pathogen entry moiety, wherein the mouse is infected with a pathogen comprising a surface moiety that binds to the pathogen entry moiety.

33. A method comprising delivering to an immunodeficient mouse a pathogen comprising a surface moiety that binds to a pathogen entry moiety, wherein the mouse is engrafted with human cancer cells comprising the pathogen entry moiety.

34. A method comprising:
preparing a single cell suspension of human patient-derived xenograft (PDX) cells obtained from a mouse, wherein the human PDX cells comprise human cells comprising a pathogen entry moiety;
delivering to an immunodeficient mouse a sample of the single cell suspension; and
delivering to the immunodeficient mouse a pathogen comprising a surface moiety that binds to the pathogen entry moiety.

35. The method of paragraph 34, wherein the single cell suspension has been purified to remove mouse cells.

36. The method of paragraph 34 or 35, wherein the sample of the single cell suspension is delivered by tail vein injection, cardiac injection, caudal artery injection, cranial injection, hepatic artery injection, femoral injection, peritoneal injection, or tibial injection.

37. The method of any one of paragraphs 34-36 further comprising delivering to the mouse a therapeutic agent or a prophylactic agent.

38. The method of paragraph 37 further comprising assessing toxicity of the agent.

39. The method of paragraph 37 or 38 further comprising assessing efficacy of the agent for treating or preventing infection by the virus and/or development of a disease caused by the pathogen.

40. The method of any one of the preceding paragraphs further comprising assessing one or more of the following:
viral load in the mouse;
respiratory function and/or cardiac function of the mouse;
the human immune cell-mediated response to the virus; and
a biomarker of viral disease progression.

41. A collection of immunodeficient mice, wherein
each mouse of the collection comprises mouse tissue engrafted with a human patient-derived xenograft (PDX) cells from a different primary tumor, and
the human PDX cells of each mouse of the collection express a combination of host cell moieties, wherein expression levels of the host cell moieties vary among mice of the collection.

42. The collection of immunodeficient mice of paragraph 41, wherein each mouse is infected with a pathogen.

### EXAMPLES

### Example 1

### Patient-Derived Xenograft (PDX) Model Selection and Growth

Using the expression data available on the JAX Mouse Tumor Biology Database (MTB), relative expression levels of ACE2, CD147 and TMPRSS2 across all PDX models were compared (Tables 1 and 2). Expression levels of pathogen entry proteins were found to vary among PDX models. Tables 1 and 2 list the PDX model identifier, the site where the tumor sample used to establish the PDX was isolated, the tumor tissue of origin, and z-based expression scale of genes required for entry of SARS-CoV-2.

Additionally, each model was assigned a tumor mutational burden (TMB) score. It is important to select PDX models with low TMB scores since it decreases the likelihood that unidentified key factors are mutated. Additionally, a low TMB score suggests better genetic stability and thus increased reproducibility from cohort to cohort. The PDX models have also been characterized by their growth kinetics. Therefore, models that do not take prohibitively long to establish tumors, but that also permit enough time for infection/treatment studies before the animal reaches a cancer endpoint. As COVID-19 presents with respiratory symptoms, PDX models derived from lung cancers were used. A recent study found differential expression of ACE2, TMPRSS2 and FURIN across cell types in the lung and identified transient secretory cells as highly vulnerable for SARS-CoV-2 infection (3). While FURIN is not directly measured by RNAseq, a surrogate marker for FURIN (CD147) has been identified and data suggest FURIN levels correlate to that of-ACE2 and T1PRSS2. However, models originating from other tissues may be used if relevant protein expression combinations are present. All RNAseq expression data was confirmed using immunohistochemistry (IC). To propagate the necessary PDX tissue, tumor fragments (either fresh or cryo-preserved) were subcutaneously implanted into the rear flank of NSG™ mice (NOD.CgPrkdC^{scid}Il2^{tm1Wj1}/SzJ, JAX strain #005557). Tumor growth was monitored weekly using caliper measurements and resulting tumors were dissected and serially passaged as needed to generate sufficient donor tissue for subsequent intravenous (IV) injection.

TABLE 1
Chosen PDX Models and Z-based Expression Scales of Genes with Known Connection to COVID-19

| | Specimen | Primary | | | | |
|---|---|---|---|---|---|---|
| Model | Site | Site | ACE2 | CD147 | TMPRSS2 | Rationale |
| TM00212 | Lung | Lung | −0.49 | 1.46 | −1.26 | Low ACE2 & TMPRSS2, High CD147 |
| J000100672 | Lung | Lung | −0.40 | 1.01 | 0.47 | Low ACE2, High CD147, Normal TMPRSS |
| TM01031 | Lung | Lung | −0.11 | 0.20 | 0.11 | Similar to normal tissue |
| J000102680 | Liver | Colon | 0.03 | 1.30 | 0.68 | Normal ACE2 & TMPRSS, High CD147 |
| TM00186 | Lung | Lung | 0.04 | 0.13 | 1.26 | Normal ACE2 & CD147, High TMPRSS2 |
| TM01510 | Lung | Lung | 0.10 | 0.24 | 0.05 | Similar to normal tissue |
| TM00355 | Lung | Lung | 0.83 | 0.77 | −1.34 | High ACE2 & CD147, Low TMPRSS2 |
| TM00219 | Lymph node | Lung | 1.93 | −0.92 | 1.06 | High ACE2 & TMPRSS2, Low CD147 |
| TM00388 | Lung | Rectum | 2.48 | −0.16 | 1.74 | High ACE2 & TMPRSS2, Normal CD147 |
| TM00134 | Lung | Colon | 2.81 | 0.30 | 1.64 | High ACE2 & TMPRSS2, Normal CD147 |

TABLE 2
Additional Chosen PDX Models and Z-based Expression Scales
of Genes with Known Connection to COVID-19

| | Specimen | Primary | | | |
|---|---|---|---|---|---|
| Model | Site | Site | ACE2 | CD147 | TMPRSS2 |
| TM000302 | Lung | Lung | −1.41 | 0.25 | −0.01 |
| TM00233 | Lung | Lung | −0.72 | −0.92 | −0.09 |
| TM00194 | Lung | Pleural | −0.59 | −1.87 | −0.88 |
| | | Cavity | | | |
| J000100672 | Lung | Lung | −0.54 | 0.55 | 0.35 |
| J000100672 | Pleural | Lung | −0.54 | 0.55 | 0.35 |
| | Cavity | | | | |
| TM00212 | Lung | Lung | −0.49 | 1.46 | −1.26 |
| J000102680 | Liver | Colon | −0.33 | 1.49 | −0.31 |
| TM01031 | Lung | Lung | −0.23 | 0.40 | −0.42 |
| TM00186 | Lung | Lung | −0.12 | 0.26 | 1.59 |
| TM01510 | Lung | Lung | 0.05 | 0.30 | 0.14 |
| TM000192 | Lung | Lung | 0.14 | 0.26 | 0.15 |
| J000108200 | Lung | Lung | 0.4 | −0.49 | 0.78 |
| TM00256 | Lung | Lung | 0.42 | 0.08 | −0.22 |
| TM00355 | Lung | Lung | 0.48 | 1.05 | −1.60 |
| TM01533 | Lung | Lung | 0.48 | 0.36 | −0.63 |
| TM00263 | Lung | Lung | 0.7 | −0.33 | 0.32 |
| TM00170 | Colon | Liver | 0.79 | 0.47 | 1.43 |
| TM00214 | Lung | Lung | 0.8 | 0.12 | −0.21 |
| TM00877 | Lung | Lung | 0.85 | 0.09 | 0.76 |
| TM00849 | Rectum | Liver | 0.92 | 0.34 | 1.07 |
| J000115420 | Lung | Lung | 1.17 | −0.90 | 0.00 |
| TM00219 | Lymph | Lung | 1.93 | −0.92 | 1.06 |
| | node | | | | |
| TM00388 | Lung | Rectum | 2.48 | −0.16 | 1.74 |
| TM00134 | Lung | Colon | 2.77 | 0.61 | 1.63 |

### PDX Cell Isolation and Re-engraftment Into Mice

Mice bearing donor PDX tissue were euthanized via CO₂ and cervical dislocation and the tumors were resected. The tumors were cleaned of necrotic, fibrous or damaged tissue and then minced into fragments smaller than 2 mm×2 mm. Tumor dissociation was achieved using a Human Tumor Dissociation Kit and gentleMACS™ dissociator (both from Miltenyi Biotec). The manufacturer's recommended protocol was followed. The resulting cell suspension was filtered with a cell strainer to remove chunks and centrifuged to remove cell debris. A Miltenyi MultiMACS cell separator was used in conjunction with a Mouse Cell Depletion Kit (Miltenyi Biotec) following the manufacturer's recommended protocol. The result was a single cell suspension of PDX tumor cells that has been depleted of any mouse stromal or endothelial cells. These cells were counted using a TC20 automated cell counter (BioRad) and viability was determined by Trypan Blue staining. If viability was less than 80%, dead cells and cellular debris was removed using Ficoll-Paque density gradient centrifugation. Cells were then be re-suspended in sterile PBS and up to 5×10⁶ cells were injected into the tail vein of NSG™ MHC II KO mice (NOD.Cg-Prkdc^{scid} H2-Ab1^{em1Mvw} H2-K1^{tm1Bpe} H2-D1^{tm1Bpe} Il12rg^{tm1Wj1}/SzJ, JAX strain #025216). This particular NSG™ variant is the most resistant to xenogeneic graft versus host disease (GVHD) and thus permits the largest window of time to conduct a study after PBMC humanization.

### Validation of Lung Engraftment

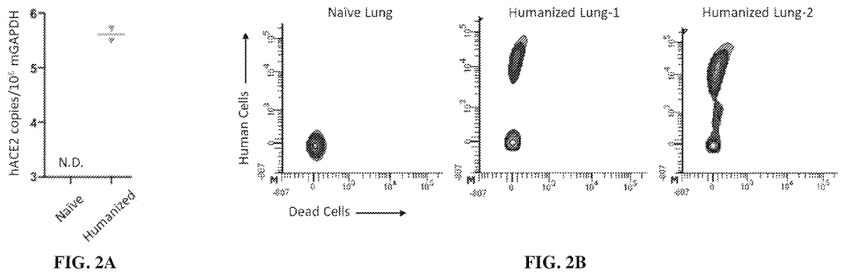

At fixed times post tail-vein injection of the PDX derived cells, a single cohort mouse was sacrificed and both lungs were removed. One lung was formalin-fixed and paraffin embedded to generate a block for histology. The relative burden of human cells was detected by using IHC to detect hKi67. This process ensures the tumors growing are of human origin rather than mouse. Simultaneously, the other lung will be collected in media. This lung was dissociated and stained for human and mouse HLA and markers such as human ACE2 (hACE2) along with lineage markers such as CD31 (endothelial), EpCAM (epithelia) and CD45 (hematopoietic). The relative burden of human cells versus mouse cells was calculated and compared to the IHC results. As shown in FIG. 2A, qPCR was used to detect hACE2 mRNA in the lungs of mice injected with PDX cells (n=2 mice per group; experiment repeated three times). Human ACE2 (hACE2) was not detected in naïve mice and was detected in the humanized mice. The signals were normalized to mouse GAPDH mRNA. Flow cytometry was performed on cells isolated from the mouse lungs (humanized or naïve mice) to analyze the presence of human cell surface proteins (HLA-ABC, CD29, and EpCAM). As shown in FIG. 2B, human cells were found in the humanized lung cell samples (rightmost two graphs) and were not found in the naïve lung cell sample (left graph), confirming that mouse lungs can be humanized by engrafting with cells for PDXs.

Humanization with Peripheral Blood Mononuclear Cells (PBMCs)

While not all COVID-19 studies will require it, the ability to engraft human immune cells is important for this model. Once engraftment of the PDX tissue is confirmed and the PDX models which provide strong viral targeting in the lung are identified, the mice will receive 0.1-5×10⁷ human PBMCs injected IV. To prevent the human T-cells from mounting a response against the PDX tissue, we will only use donor PBMCs with matched MHC-I (and MHC-II if possible). Depending on the number of cells injected, blood samples will be collected via retro-orbital bleeding up to 30 days post PBMC injection. Flow cytometry will be used to analyze this blood for human immune cells and determine the engraftment rate.

### Infection and Clinical Observations

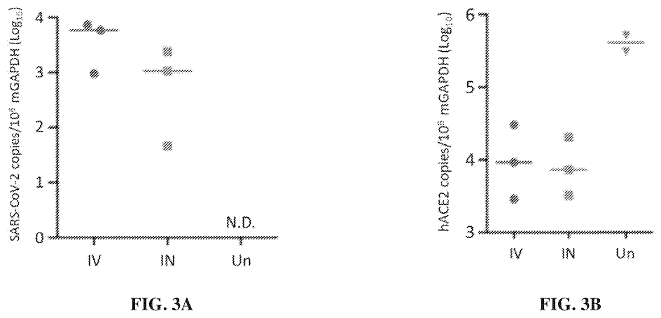

Infection of tumors was confirmed in vitro. PDX models that support SARS-CoV-2 infection were engrafted into mice and infected where infection was monitored by viral load and clinical outcome. Mice (n=3 per group) were infected intranasally (IN) or intravenously (IV) with SARS-CoV-2 (2×10⁴ FFU). Two days after infection, the mice were harvested and analyzed. FIG. 3A shows that both routes of infection resulted in the presence of detectable SARS-CoV-2 mRNA in PDX-humanized mouse lungs, as measured by qPCR. Uninfected mice (Un) did not have any detectable levels of SARS-CoV-2 mRNA. Levels of hACE2 were also quantified (FIG. 3B) and were found to be lower in the lungs of PDX-humanized mice infected with SARS-CoV-2 compared to in uninfected mice, indicating that hACE2-positive PDX cells die or that their gene expression decreases after expression. Therefore, PDX-humanized mice are susceptible to multiple routes of infection. The signals in both graphs were normalized to mouse GAPDH mRNA.

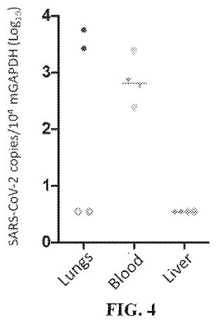

The tissues involved in SARS-CoV-2 infections were examined using a PDX-humanized mouse model. PDX-humanized mice and naïve mice (n=2 per group; repeated twice) were treated with MAR-1, an antibody that blocks mouse Type 1 interferon, one day before being infected with SARS-CoV-2 intranasally (5×10⁴ FFU). Two days after infection, the mice were harvested. Then, qPCR was used to measure SARS-CoV-2 mRNA expression in the lungs, blood, and liver of the mice. The results are shown in FIG. 4, and they demonstrate that the virus was present in the bloodstream of both the PDX-humanized mice and the naïve mice but was only present in the lungs of the PDX-humanized mice (closed circles compared to open circles). As expected, the virus was not detected in the livers of either group. The signals were normalized to mouse GAPDH mRNA.

Tissue involvement was further examined using the HuH7.5-Luc humanized mouse.

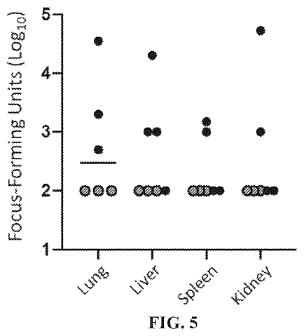

HuH7.5 cells have been demonstrated to propagate SARS-CoV-2 in vitro. Naïve and HuH7.5-Luc humanized mice (n=3 per group; repeated twice) were treated with MAR-1 one day prior to infection and then infected with SARS-CoV-2 intravenously (2×10⁵ FFU). Lungs, liver, spleen, and kidneys from the mice were harvested two days after infection and tissue homogenate was used to detect the virus. As shown in FIG. 5, a plaque assay demonstrated that all humanized tissues contained replicating virus (black circles) but there was no evidence of replication in the naïve mice (gray circles).

Subsequent humanization by injection with PBMCs (either normal or patient PBMCs containing viral particles) will be attempted to examine the effects of the human immune system on infection and viral load. Due to the clinical presentation of COVID-19, the focus will be on detecting lung and heart abnormalities. SLU has established non-endpoint methods to evaluate respiratory function (plethsmography), cardiac function (echocardiography and electrocardiography) and both (pulse oximetry). Additionally, there are several serum biomarkers that correlate with a poor prognosis that we will monitor. This includes markers for cardiovascular damage including cardiac troponin I (TnI) and brain-type natriuretic peptide (BNP) (15-17). The humanized mice will require additional characterizations post infection with SARS-CoV-2. Exaggerated systemic inflammation/cytokine storm is a hallmark of severe disease (18,19), so markers like IL-2, IL-6, TNF-α, interferon-7, and GCSF can be used. To monitor clinical progression, we will process blood from both symptomatic and asymptomatic mice into serum at defined intervals and evaluate marker levels.

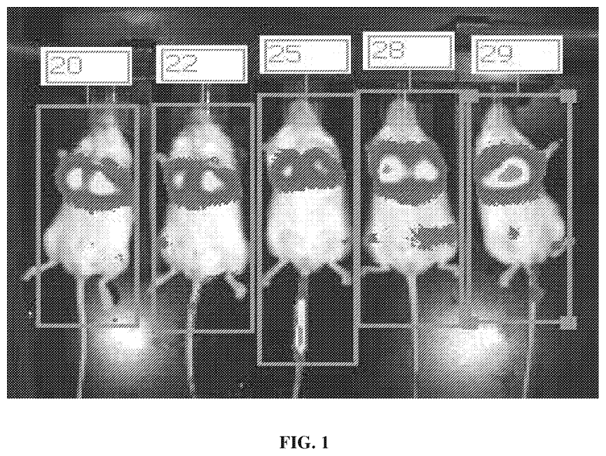

As demonstrated herein, injection into the tail vein may be used to engraft human cancer cells into mice (6,7). For most PDX models, the cancer cells colonize in the lung regardless of the tissue of origin. We have injected a luciferase-tagged breast cancer cell line into NSG™ and NSG-MHC II KO mice (NOD.Cg-Prkdc^{scid}H2-K1^{tm1Bpe}H2-Ab1^{em1Mvw}H2-D1^{tm1Bpe}Il2rg^{tm1Wj1}/SzJ; JAX Stock #025216). Using in vivo imaging systems (IVIS) to detect luciferase activity, we clearly demonstrated that the majority of the cells engraft into the lungs (FIG. 1). PDXs are models of cancer where the tissue or cells from a patient's tumor are implanted into immunodeficient mice. Once established, the PDX tissue can be propagated and re-engrafted into a large number of mice to generate study cohorts. The PDX Resource at Jackson Laboratories (JAX) has developed over 400 models from patient samples all over the United States. These models represent a variety of ages, ethnicities, tissue types, and mutational burdens. RNA sample(s) from each of the 400 models have been analyzed using RNAseq as part of model characterization, so a robust transcriptome is available.

### Example 2

### SARS-CoV-2 Infection Mouse Model

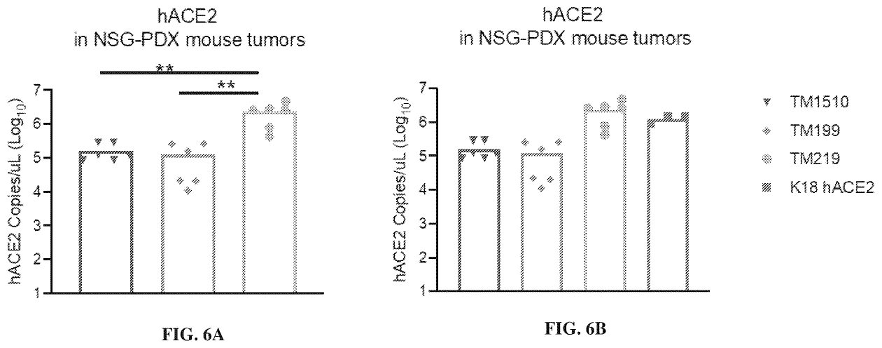

NSG mice humanized with different cell lines (TM1510, TM199, and TM219) (n=6 per group) were treated with MAR-1, an antibody that blocks mouse interferon-alpha/beta receptor, one day before being infected with SARS-CoV-2 AZ1 (isolate USA-AZ1/2020) intranasally (5×10⁴ FFU). As a baseline, qPCR was used to measure expression levels of human ACE2 in the humanized mice's tumors two or four days after injection with MAR-1. The results are shown in FIGS. 6A-6B and demonstrate that the NSG-PDX TM219 mice had significantly higher hACE2 expression levels relative to the other two cell lines tested (TM1510 and TM199) (FIG. 6A). Notably, the amount of hACE2 expression observed in the TIM219 mice was similar to the hACE2 expression seen in lungs isolated from k18 transgenic mice (K18-ACE2 mouse), which are known to be susceptible to SARS-CoV-2 (FIG. 6B).

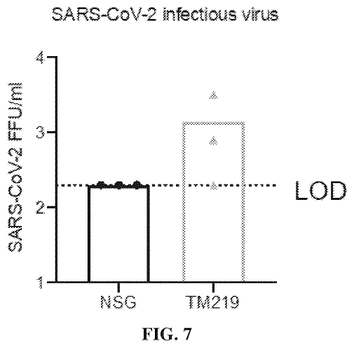

In a further experiment, NSG mice humanized with TM219 or NSG mice (as a control) (n=3 per group) were treated with MAR-1, one day before being infected with SARS-CoV-2 AZ1 intranasally (5×10⁴ FFU). Two days later, the number of focus forming units (FFU) per milliliter was measured to determine the level of SARS-CoV-2 infection in the lungs of the mice. As can be seen in FIG. 7, SARS-CoV-2 infectious virus was detected in two of three NSG PDX TM219 mice and none of the NSG control mice. All of the NSG control mice had levels below the limit of detection.

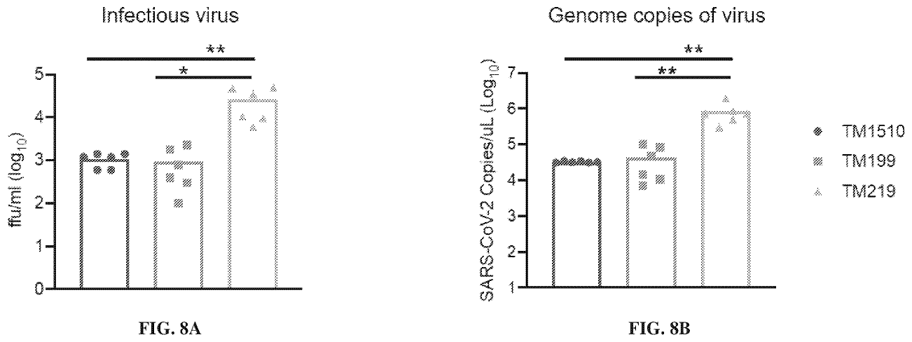

The SARS-CoV-2 infectious virus and genome copies of the virus were compared between different PDXs. The NSG mice were humanized with different cell lines (TM1510, TM199, and TM219) (n=6 per group) and then treated with MAR-1, one day before being infected with SARS-CoV-2 AZ1 intranasally (5×10⁴ FFU). Two days later, the number of genome copies of SARS-CoV-2 and the FFU/mL were measured (FIGS. 8A-8B). The NSG-PDX TM219 line had significantly levels of SARS-CoV-2 RNA copies (FIG. 8B) and infectious virus (FIG. 8A) at 48 hours post infection relative to TM1510 and TM199. This result correlates with the higher hACE2 expression seen in the TM219 line (FIG. 6A). Therefore, the TM219 line is better than the other tumor lines tested at supporting viral replication. In addition, there appears to be a link between hACE2 expression and level of infection.

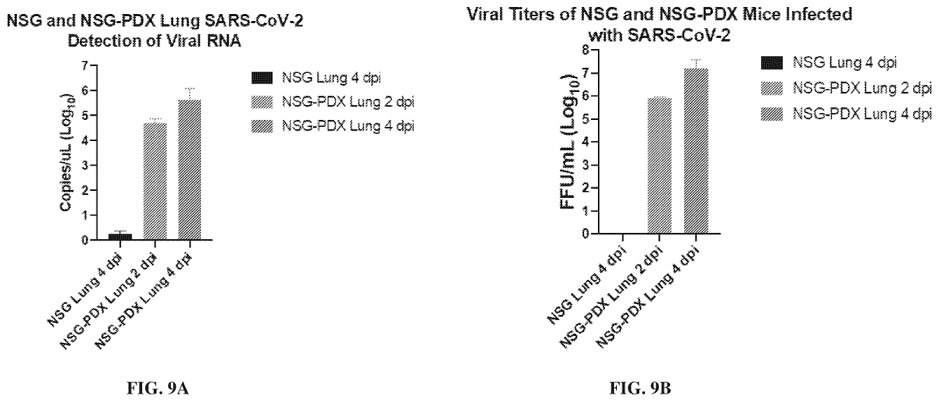

A time course experiment was undertaken to explore the viral titer and viral load of SARS-CoV-2 in the lungs of NSG (control) and NSG-PDX mice at two and four days post infection. Briefly, NSG mice were humanized with TM219 and then both groups (NSG and NSG-PDX) were treated with MAR-1 one day before being infected with SARS-CoV-2 AZ1 intranasally (5×10⁴ FFU). Two and four days later, the viral load (FIG. 9A) and viral titer (FIG. 9B) were measured in lung tissue. The NSG mice showed no replication of active infectious SARS-CoV-2 virus in lung tissue as well as very low detection of viral RNA found in lungs compared to the NSG-PDX mice expressing tumor 219 cells in the lung at both time points. There was also a log difference between timepoints for the NSG-PDX group showing both infectious virus (FFA) and viral RNA load of SARS-CoV-2 in the lungs.

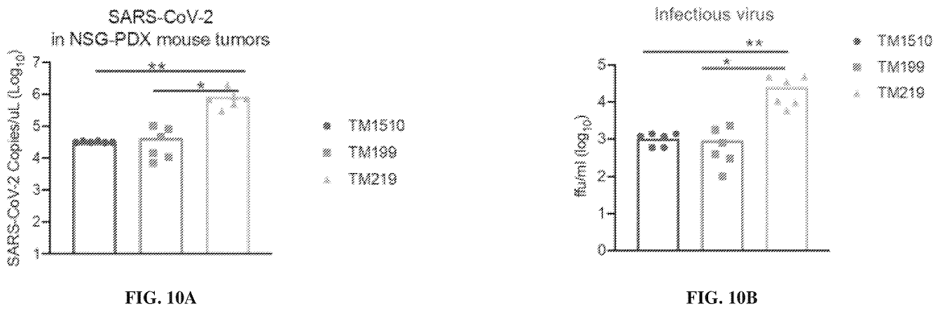

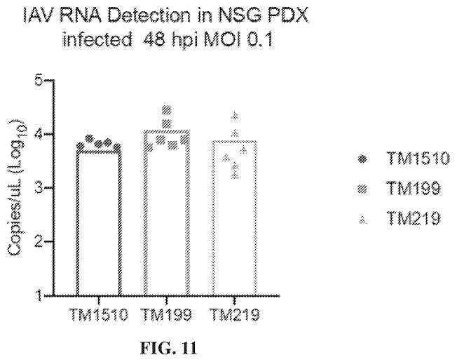

An in vitro experiment was undertaken to further study infections in PDX. Briefly, NSG mice were humanized with different cell lines (TM1510, TM199, TM219, TM 1031, and TM1446). The resulting tumors were removed and homogenized. Then, 1×10⁶ cells were plated per well into three wells of a three 12 well plates. Each of the three 12 well plates then contained three wells of cells from all four tumor lines. Plate 1 was infected with SARS-CoV-2 with 1×10⁶ infectious particles per well. Plate 2 was infected with influenza A (IAV) with 1×10⁵ infectious particles per well. Plate 3 contained media with the addition of PBS. Supernatant was taken at the time of infection and at 48 hours. RNA was isolated from cells at 48 hours. The supernatant at 48 hours was also used for focus forming assays to detect infectious virus. The experiment was performed, in triplicate, twice. The results from SARS-CoV-2 infection are shown in FIGS. 10A-10B and demonstrate that TM219 had significantly higher levels of SARS-CoV-2 mRNA at 48 hours compared to TM199, and TM1510 (FIG. 10A) and demonstrated that the TM219 line is better than other tumor lines at supporting viral replication (FIG. 10B). This correlates with the higher hACE2 expression seen in the TM219 line. With respect to influenza A, there were no differences between the levels of influenza A mRNA expressed in the NSG-PDX lines mRNA at 48 hours (FIG. 11). As the influenza A virus binds to a modification of protein on the surface of human cells, no difference in the genetically different PDXs was expected.

### Example 3

### In Vitro Infection of Primary Cell Lines

To understand the potential variation in virus susceptibility in three different patient-derived explants, PDX samples were infected with four different viruses that represent three different viral families. These viruses are representative examples of emerging viral infections present within the Western hemisphere.

Briefly, patient-derived explants were digested and used as single cells suspensions. The three explants were TM01031 (lung adenosquamous carcinoma), TM01446 (lung adenocarcinoma), and TM00219 (lung adenocarcinoma). These single cell suspensions were plated into 96 well plates and then infected at a multiplicity of infection (MOI) of 0.5 (multiplicity of infection) with a panel of viruses representing three different virus families. In particular, three Flaviviradae were tested: West Nile virus (WNV), Langat virus (LGTV), and two strains of Powassan virus (POWV): LB and Spooner; one Togaviradae was tested: Chikungunya (CHIKV); and one Bunyaviradae: LaCrosse virus (LACV) was tested. One hour after infection, the cells were washed once to remove cell-free and surface bound virus, and then resuspended in media. The virally infected cells were sampled over time at the time of infection (following the wash step), 24 hours later, and 48 hours later. Total RNA was isolated from infected cells and qRT-PCR was performed. The assay also measured a housekeeping gene (TaqMan Human GapDH) and a virus-specific primer probe (TaqMan primer probes). Note that uninfected cells were also harvested at each time point as a control and showed no difference in total cell number compared to infected (data not shown).

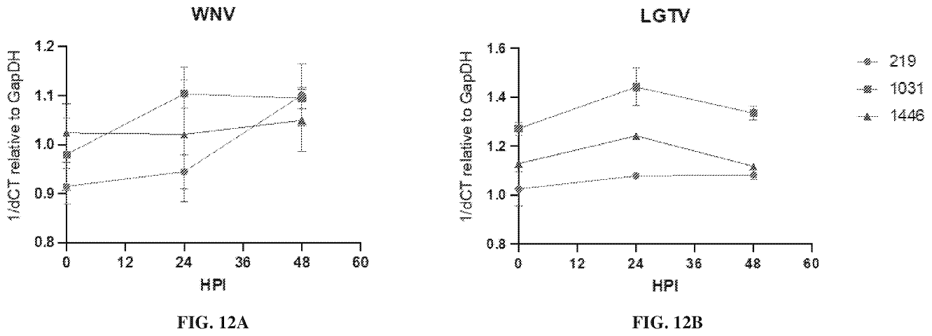

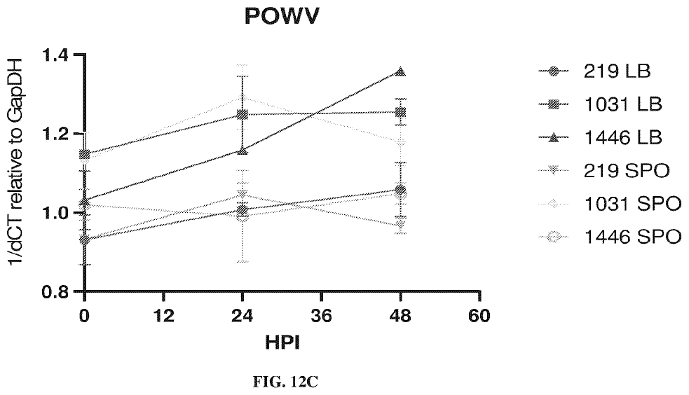

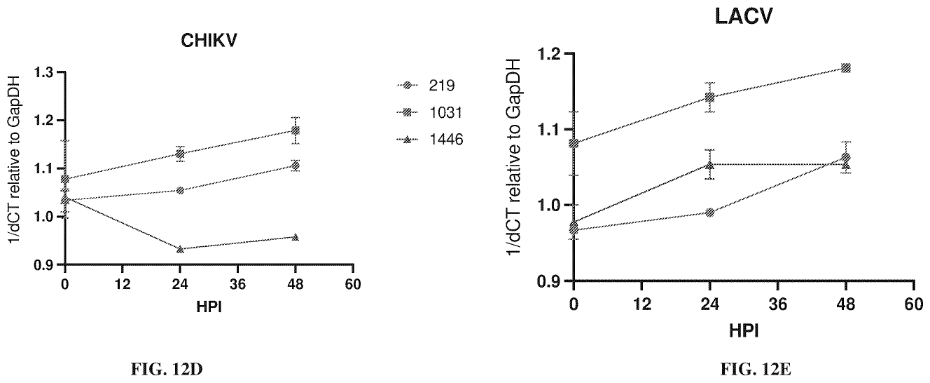

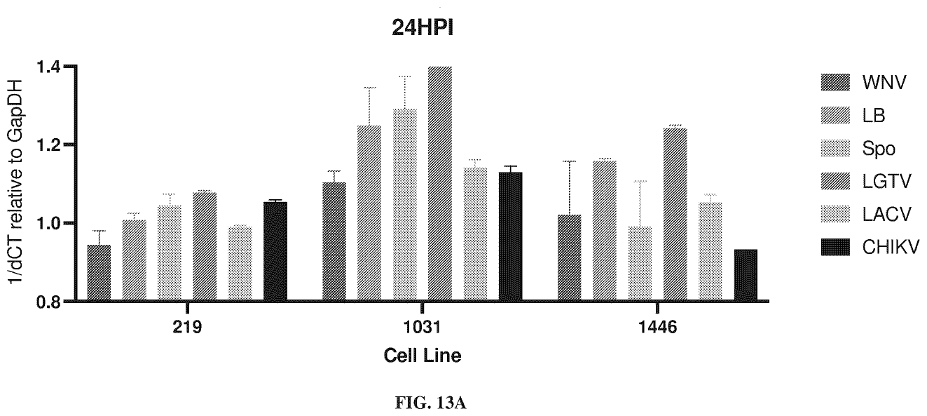

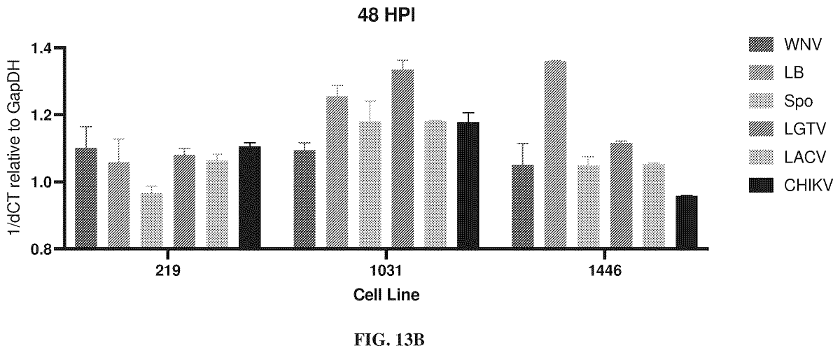

The results are shown in FIGS. 12A-12E (viral RNA levels) and FIGS. 13A-13B (levels at 24 hours post-infection and 48 hours post-infection, respectively). With respect to WNV, the three PDX samples resulted in two phenotypes: in 1031 and 0219, WNV viral RNA increased in the PDX samples, with 1031 potentially showing an increase in viral RNA at 24 hours while 0219 had an increase in viral RNA from 24-48 hours (FIG. 12A). In contrast, 1446 did not support an increase in WNV viral RNA load. For LGTV, which is not known to infect humans, this virus did not increase in viral RNA load in any of the three PDX samples, replicating the anticipated results (FIG. 12B). With respect to POWV, the LB infection viral load increased in 1446 and 0219, while 1031 did not appear to support viral replication (FIG. 12C). For the SPO strain, it does not appear that any of the three PDX samples support viral RNA replication (FIG. 12C). For CHIKV, both 0219 and 1031 appear to support viral RNA replication, while 1446 does not appear to support viral RNA replication (FIG. 12D). All three PDX samples could support limited LACV virus RNA replication (FIG. 12E).

As shown in FIGS. 13A-13B, 1031 was capable of supporting 5/6 virus infections except for SPO. Viral RNA increases in 1031 predominantly occurred over the first 24 hours. 0219 was capable of supporting 4/6 viral infections, with increases occurring at both the 24 and 48 hour times points, with the exception of LGTV, and SPO viruses, while 1446 only supported viral RNA replication of POWV LB (1/6 viruses). Not shown—uninfected cells were also harvested at each time point and showed no difference in total cell number compared to infected.

### REFERENCES

1. Walls A C, Park Y J, Tortorici M A, Wall A, McGuire A T, Veesler D. Structure, Function, and Antigenicity of the SARS-CoV-2 Spike Glycoprotein. Cell. 2020. doi:10.1016/j.cell.2020.02.058
2. Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell. 2020. doi:10.1016/j.cell.2020.02.052
3. Lukassen S, Chua R L, Trefzer T, et al. SARS-CoV-2 receptor ACE2 and TMPRSS2 are predominantly expressed in a transient secretory cell type in subsegmental bronchial branches. bioRxiv. 2020. doi:10.1101/2020.03.13.991455
4. Wang K, Chen W, Zhou Y-S, et al. SARS-CoV-2 invades host cells via a novel route: CD147-spike protein. bioRxiv. 2020. doi:10.1101/2020.03.14.988345
5. Asselta R, Paraboschi E M, Mantovani A, Duga S., ACE2; and TMPRSS2 variants and expression as candidates to sex and country differences in COVID-19 severity in Italy. medRxiv. 2020. doi:10.1101/2020.03.30.20047878
6. Yang S, Zhang J J, Huang X-Y. Mouse Models for Tumor Metastasis, Rational Drug Design; 2012. doi:10.1007/978-1-62703-008-3_17
7. Rashid O M, Nagahashi M, Ramachandran S, et al. Is tail vein injection a relevant breast cancer lung metastasis model? J Thorac Dis. 2013. doi:10.3978/j.issn.2072-1439.2013.06.17
8. Keck J G, Makino S, Soe L H, Fleming J O, Stohlman S A, Lai M M. RNA recombination of coronavirus. Adv Exp Med Biol. 1987. doi:10.1007/978-1-4684-1280-2_11
9. Keck J G, Matsushima G K, Makino S, et al. In vivo RNA-RNA recombination of coronavirus in mouse brain. J Virol. 1988. doi:10.1128/jvi.62.5.1810-1813.1988
10. Lai M M, Baric R S, Makino S, et al. Recombination between nonsegmented RNA genomes of murine coronaviruses. J Virol. 1985. doi:10.1128/jvi.56.2.449-456.1985
11. Keck J G, Hogue B G, Brian D A, Lai M M C. Temporal regulation of bovine coronavirus RNA synthesis. Virus Res. 1988. doi:10.1016/0168-1702(88)90093-7
12. Lai M M C, Makino S, Soe L H, Shieh C K, Keck J G, Fleming J O. Coronavirus: A jumping RNA transcription. Cold Spring Harb Symp Quant Biol. 1987. doi:10.1101/SQB.1987.052.01.041
13. Draheim K M, Chen H-B, Tao Q, Moore N, Roche M, Lyle S. ARRDC3 suppresses breast cancer progression by negatively regulating integrin B4. Oncogene. 2010; 29(36). doi:10.1038/onc.2010.250
14. Moore N, Houghton J, Lyle S. Slow-cycling therapy-resistant cancer cells. Stem Cells Dev. 2012. doi:10.1089/scd.2011.0477
15. Guo T, Fan Y, Chen M, et al. Cardiovascular Implications of Fatal Outcomes of Patients with Coronavirus Disease 2019 (COVID-19). JAMA Cardiol. 2020. doi:10.1001/jamacardio.2020.1017
16. Zhou F, Yu T, Du R, et al. Clinical course and risk factors for mortality of adult inpatients with COVID-19 in Wuhan, China: a retrospective cohort study. Lancet. 2020. doi:10.1016/S0140-6736(20)30566-3
17. Arentz M, Yim E, Klaff L, et al. Characteristics and Outcomes of 21 Critically Ill Patients with COVID-19 in Washington State. JAMA—J Am Med Assoc. 2020. doi:10.1001/jama.2020.4326
18. Mehta P, McAuley D F, Brown M, Sanchez E, Tattersall R S, Manson J J. COVID-19: consider cytokine storm syndromes and immunosuppression. Lancet. 2020. doi:10.1016/S0140-6736(20)30628-0
19. Siddiqi H K, Mehra M R. COVID-19 Illness in Native and Immunosuppressed States: A Clinical-Therapeutic Staging Proposal. J Hear Lung Transplant. 2020. doi:10.1016/j.healun.2020.03.012

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. An immunodeficient mouse engrafted with cells comprising a host cell moiety associated with pathogenesis, wherein the mouse is infected with a pathogen comprising a surface moiety that binds to the host cell moiety.

2-32. (canceled)

33. A method comprising:
administering to an immunodeficient mouse cells of a human patient-derived xenograft (PDX), wherein the cells comprise a pathogen entry moiety; and
administering to the immunodeficient mouse a pathogen comprising a surface moiety that binds to the pathogen entry moiety.

34. The method of claim 33, wherein the cells are administered sample of the single cell suspension is delivered by

35. The method of claim 33, wherein the administering is by tail vein injection, cardiac injection, caudal artery injection, cranial injection, hepatic artery injection, femoral injection, peritoneal injection, or tibial injection.

36. The method of claim 33, further comprising delivering to the mouse a therapeutic agent or a prophylactic agent.

37. The method of claim 36, further comprising assessing toxicity of the agent.

38. The method of claim 36, further comprising assessing efficacy of the agent for treating or preventing infection by the virus and/or development of a disease caused by the pathogen.

39. The method of claim 33 further comprising assessing one or more of the following:
viral load in the mouse;
viral titer in the mouse;
respiratory function and/or cardiac function of the mouse;
the human immune cell-mediated response to the virus; and
a biomarker of viral disease progression.

40. A method comprising:
infecting multiple human cell samples with a pathogen, wherein each of the samples comprises human cells from a different source;
measuring viral load for each of the samples; and
identifying a sample having a viral load greater than a reference value.

41. The method of claim 40, wherein the different sources are different patient-derived xenografts (PDXs).

42. The method of claim 40, wherein the different sources are different human subjects.

43. The method of claim 40, wherein the different sources are different cell lines.

44. The method of claim 40, further comprising delivering to an immunodeficient mouse cells of the sample having a viral load greater than a reference value.

45. The method of claim 40 further comprising assessing genetic differences among the human cells of the samples.

46. The method of claim 45, further comprising genetically mapping genes of the human cells necessary for pathogenic infection.

47. The method of claim 45, further comprising identifying at least one pathogen entry moiety used by the pathogen for entry into the human cells.

48. The method of claim 40, further comprising administering to the immunodeficient mouse the pathogen.

49. The method of claim 48, further comprising administering to the immunodeficient mouse a therapeutic agent or a prophylactic agent.

50. The method of claim 40, wherein the method is performed in vitro.
